SEQUENCE LISTING (31 sequences)

SEQ ID NO: 1 - DNA, 521 bp, synthetic construct
  misc_feature 1..521: note = sfGFPn Y66
  source 1..521: mol_type = other DNA
atggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 2 - DNA, 196 bp, synthetic construct
  misc_feature 1..196: note = sfGFPn
  source 1..196: mol_type = other DNA
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacct

SEQ ID NO: 3 - DNA, 660 bp, synthetic construct
  misc_feature 1..660: note = eYGFPuv
  source 1..660: mol_type = other DNA
atgacaaccttcaaaatcgagtcccggatccacggcaacctcaacggggagaagttcgagttggttggaggtggagtaggtgaggagggtcgcctcgagattgagatgaagactaaagataaaccactggcattctctcccttcctgctgaccacttgcatgggttacgggttctaccacttcgccagcttcccaaaggggattaagaacatctatcttcatgctgcaacgaacggaggttacaccaacaccaggaaggagatctatgaagacggcggcatcttggaggtcaacttccgttacacttacgagttcaacaagatcatcggtgacgtcgagtgcattggacatggattcccaagtcagagtccgatcttcaaggacacgatcgtgaagagttgtcccacggtggacctgatgttgccaatgtccgggaacatcatcgccagctcctacgcttacgccttccaactgaaggacggctctttctacacggcagaagtcaagaacaacatagacttcaagaatccaatccacgagtccttctcgaagtcagggcccatgttcacccacagacgtgtcgaggagactctcaccaaggagaaccttgccatagtggagtaccagcaggttttcaacagcgccccaagagacatgtag

SEQ ID NO: 4 - DNA, 678 bp, synthetic construct
  misc_feature 1..678: note = Red Fluorescent Protein
  source 1..678: mol_type = other DNA
atggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 5 - DNA, 346 bp, synthetic construct
  misc_feature 1..346: note = Cauliflower mosaic virus 35S Promoter Region
  source 1..346: mol_type = other DNA
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaaca

SEQ ID NO: 6 - DNA, 387 bp, Tetrahymena thermophila
  source 1..387: mol_type = other DNA
aaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 7 - DNA, 387 bp, synthetic construct
  misc_feature 1..387: note = dRybozyme
  source 1..387: mol_type = other DNA
aaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacaaactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 8 - DNA, 57 bp, synthetic construct
  misc_feature 1..57: note = P2A Self Cleaving Peptide
  source 1..57: mol_type = other DNA
gctactaacttcagcctgctgaagcaggctggagacgtggaggagaaccctggacct

SEQ ID NO: 9 - DNA, 41 bp, synthetic construct
  misc_feature 1..41: note = Hairpin 14
  source 1..41: mol_type = other DNA
acgtcgactctcgagtgagattgttgacggtaccgtatttt

SEQ ID NO: 10 - DNA, 20 bp, synthetic construct
  misc_feature 1..20: note = P1 Loop
  source 1..20: mol_type = other DNA
aaatagcaatatttaccttt

SEQ ID NO: 11 - DNA, 11 bp, synthetic construct
  misc_feature 1..11: note = P2 Loop_02 Fragment
  source 1..11: mol_type = other DNA
tatttaccttt

SEQ ID NO: 12 - DNA, 2137 bp, Arabidopsis thaliana
  source 1..2137: mol_type = other DNA
gagcttggtatattgatcataccaaagtggtagtgatttattgattaacccaaacacaaaataaacagatttgactcaaaaagaagaaaatgaattctaacaactggcttggctttcctctttcaccgaacaactcttctttgcctcctcatgaatacaaccttggcttggtcagcgaccatatggacaacccttttcaaacacaagagtggaatatgatcaatccacacggtggaggaggagatgaaggaggagaggttccaaaagtggccgattttctcggtgtgagcaaaccggacgaaaaccaatccaaccacctagtagcttacaacgactcagactactacttccataccaatagcttgatgcctagcgtccaatcaaacgatgtcgttgtagcagcttgtgactccaatactcctaacaacagtagctatcatgagcttcaagagagtgctcacaatctacagtcacttactttgtccatggggaccaccgctggtaataatgttgtagacaaagcttcaccatccgagaccaccggggataacgctagcggtggagcactagccgttgttgagacggccacgccaagacgtgcattggacactttcggacaacgaacctcgatctatcgtggtgtcacaagacatcgatggactggtcgatatgaggctcatctatgggataatagttgtagaagggaaggccagtctaggaaaggaagacaagtttacttgggtggatatgacaaagaagataaagcagcaagatcatatgatctagctgcacttaagtactggggtccttcaactactactaatttccccattacaaactacgagaaagaagtagaggaaatgaagcacatgacgagacaagagttcgtggctgccattagaaggaaaagtagtggattttcgagaggcgcttcgatgtatcgaggagttacaaggcatcaccaacatggaagatggcaagcaaggatcggccgagtcgccggaaacaaagacctctacttgggaacttttagcactgaggaagaagcagcagaagcttacgatatagctgcaataaagtttagaggacttaatgcagtgaccaacttcgagatcaaccggtacgacgtgaaagccattctagagagtagcactcttcccatcggaggaggcgcagctaaacggctcaaagaagctcaagctcttgagtcttcaaggaaacgcgaggcggagatgatagcccttggttcaagtttccagtacggtggtggctcgagcacaggctctggctccacctcatcaagacttcagcttcaaccttaccctctaagcattcaacaaccattagagccttttctatctcttcagaacaatgacatctctcattacaacaacaacaatgctcacgattcctcctcttttaatcaccatagctatatccagacacaacttcatctccaccaacagaccaacaattacttgcagcaacagtcgagccagaactctcagcagctctacaatgcgtatcttcatagcaatccggctctgcttcatggacttgtctctacctctatcgttgacaacaataataacaatggaggctctagtgggagctacaacactgcagcatttcttgggaaccacggtattggtattgggtccagctcgactgttggatcgaccgaggagtttccaaccgttaaaacagattacgatatgccttccagtgatggaaccggagggtatagtggttggaccagtgagtctgttcaggggtcaaaccctggtggtgttttcactatgtggaatgagtaaacaaggatctctttcttgcggcacaaggaatgggtctgaatagtaactattttaatctctactctcaatttttatttctttcttgtcttggaagtaataggatcaatttagagggggccttttggtagaagaatttaggtgacgcaaggagttctttctatatttacggcgaaggtattctcagactggttaaatggttcttgagggttagggttttatgggtactcaattaagtttgtgtacccaaattttgtttacagctaacccttttgactaatttccaaatttgtaattcgtttataaagatgttttctctagctctt

SEQ ID NO: 13 - DNA, 612 bp, synthetic construct
  misc_feature 1..612: note = tobacco rattle virus Cap protein
  source 1..612: mol_type = other DNA
atgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccc

SEQ ID NO: 14 - DNA, 420 bp, synthetic construct
  misc_feature 1..420: note = Anti-CRISPR AcrIIA5
  source 1..420: mol_type = other DNA
atggcctatggcaagtctcgttataattcctataggaaacgtagcttcaaccgatctaacaagcaacgtagagaatacgcacaggagatggatcgacttgagaaagcattcgaaaacctggacgggtggtacctttcctcaatgaaggatagcgcctacaaggattttggtaagtacgaaatccgattgtctaaccactcagccgacaataaatatcatgatctcgaaaatgggcgattgatcgtgaacatcaaggcttcaaaactcaattttgtcgatattatcgaaaataaacttgataagatcatcgagaagatcgacaagctcgacctggataagtatagatttattaacgcaaccaatctcgaacatgacattaagtgctactacaaaggcttcaagacgaaaaaagaggtgatt

SEQ ID NO: 15 - DNA, 41 bp, synthetic construct
  misc_feature 1..41: note = gRNA_01
  source 1..41: mol_type = other DNA
ggaggagtcctgggtaacggtaacaacaccaccgtcttcga

SEQ ID NO: 16 - DNA, 41 bp, synthetic construct
  misc_feature 1..41: note = gRNA_02
  source 1..41: mol_type = other DNA
tggtaccacgcagtttaactttgtagatgaactcaccgtct

SEQ ID NO: 17 - DNA, 82 bp, synthetic construct
  misc_feature 1..82: note = gRNA_03
  source 1..82: mol_type = other DNA
ggaggagtcctgggtaacggtaacaacaccaccgtcttcgaagttcataacacgttcccatttgaaaccttccgggaaggac

SEQ ID NO: 18 - DNA, 82 bp, synthetic construct
  misc_feature 1..82: note = gRNA_04
  source 1..82: mol_type = other DNA
catggtttttttctgcataaccggaccgtcggacgggaagttggtaccacgcagtttaactttgtagatgaactcaccgtct

SEQ ID NO: 19 - DNA, 82 bp, synthetic construct
  misc_feature 1..82: note = gRNA_05
  source 1..82: mol_type = other DNA
ccggtttgctcacaccgagaaaatcggccacttttggaacctctcctccttcatctcctcctccaccgtgtggattgatcat

SEQ ID NO: 20 - DNA, 82 bp, synthetic construct
  misc_feature 1..82: note = gRNA_06
  source 1..82: mol_type = other DNA
ttgattggacgctaggcatcaagctattggtatggaagtagtagtctgagtcgttgtaagctactaggtggttggattggtt

SEQ ID NO: 21 - DNA, 123 bp, synthetic construct
  misc_feature 1..123: note = gRNA_07
  source 1..123: mol_type = other DNA
ggaggagtcctgggtaacggtaacaacaccaccgtcttcgaagttcataacacgttcccatttgaaaccttccgggaaggacagtttcaggtagtccgggatgtcagccgggtgtttaacgta

SEQ ID NO: 22 - DNA, 123 bp, synthetic construct
  misc_feature 1..123: note = gRNA_08
  source 1..123: mol_type = other DNA
ccgtcttccgggtacatacgttcggtggaagcttcccaacccatggtttttttctgcataaccggaccgtcggacgggaagttggtaccacgcagtttaactttgtagatgaactcaccgtct

SEQ ID NO: 23 - DNA, 164 bp, synthetic construct
  misc_feature 1..164: note = gRNA_09
  source 1..164: mol_type = other DNA
ggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtccc

SEQ ID NO: 24 - DNA, 164 bp, synthetic construct
  misc_feature 1..164: note = gRNA_10
  source 1..164: mol_type = other DNA
ctttcagtttcagacgcattttgatttcacctttcagagcaccgtcttccgggtacatacgttcggtggaagcttcccaacccatggtttttttctgcataaccggaccgtcggacgggaagttggtaccacgcagtttaactttgtagatgaactcaccgtct

SEQ ID NO: 25 - DNA, 249 bp, synthetic construct
  misc_feature 1..249: note = gRNA_11
  source 1..249: mol_type = other DNA
ggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcggaccacctttggtaactttcagtttagcggtctgggtaccttcgtacggacgaccttcaccttcaccttc

SEQ ID NO: 26 - DNA, 878 bp, Arabidopsis thaliana
  source 1..878: mol_type = other DNA
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctgattagattcaccgaccctcctatcgtgtcgacctttctgtttcttagaattttttggtagtctatgtactaataatgtcagcttcgtatttatttcataagcaatttgcatttgcaatttgttttttacttttatttttattgtattgtggaatgtggactcgtaccaacatgaagttatataccaccaaaaaaattacagttagtcaaaagattcacgagtgagagctacttatgattgtcttttacgtatatgtctaattgtctatttgctcaataatctttgtactttcttttgtcgttgataaaatcacaaagttccaaaagtaatcgaatgatttgcttttaagaaaagaagagctcaataattcaacatatatctgtacaca

SEQ ID NO: 27 - DNA, 107 bp, Arabidopsis thaliana
  source 1..107: mol_type = other DNA
caaacattactcattcacaaaaccatcttaaagcaactacacaagtcttgaaattttctcatattttctatttactatataaacttttaatcaaatcaagattaaag

SEQ ID NO: 28 - protein, 19 aa, synthetic construct
  REGION 1..19: note = Teschovirus asilesi
ATNFSLLKQAGDVEENPGP

SEQ ID NO: 29 - protein, 20 aa, synthetic construct
  REGION 1..20: note = Aphthovirus burrowsi
QCTNYALLKLAGDVESNPGP

SEQ ID NO: 30 - protein, 22 aa, synthetic construct
  REGION 1..22: note = Aphthovirus vesiculae
VKQTLNFDLLKLAGDVESNPGP

SEQ ID NO: 31 - protein, 18 aa, synthetic construct
  REGION 1..18: note = Alphapermutotetravirus thoseae
EGRGSLLTCGDVEENPGP

(19) United States
(12) Patent Application Publication
(10) Pub. No.: US 2026/0071258 A1
(43) Pub. Date: Mar. 12, 2026

(54) SPLIT RIBOZYME BIOSENSOR SYSTEMS

(71) Applicant: UT-Battelle, LLC, Oak Ridge, TN (US)

(72) Inventors: Yang Liu, Ames, IA (US); Gerald A. Tuskan, Oak Ridge, TN (US); Ilenne Del Valle Kessra, Oak Ridge, TN (US); Paul E. Abraham, Knoxville, TN (US); Xiaohan Yang, Knoxville, TN (US)

(21) Appl. No.: 19/244,424

(22) Filed: Jun. 20, 2025

Related U.S. Application Data
(60) Provisional application No. 63/662,436, filed on Jun. 21, 2024.

(51) Int. Cl. C12Q 1/6823; C12N 15/113; C12N 15/52; C12N 15/82; C12Q 1/26; C12Q 1/6809; C12Q 1/6825

(52) U.S. Cl. CPC C12Q 1/6823; C12N 15/113; C12N 15/52; C12N 15/8242; C12Q 1/26; C12Q 1/6809; C12Q 1/6825; C12Y 113/12007; C12N 2830/002; C12Q 2600/13

(57) ABSTRACT

The present disclosure is directed to a split ribozyme biosensor system. Additionally, a genetically modified plant, plant tissue, or plant cell comprising the split ribozyme biosensor system is described. Methods for examining in vivo RNA expression in plants, plant cells or plant tissues are disclosed. Lastly, disclosed herein is a kit comprising the split ribozyme biosensor system.

### CROSS REFERENCE TO RELATED APPLICATIONS

This application claims the benefit of U.S. Provisional Patent Application No. 63/662,436, filed Jun. 21, 2024, the contents of which is incorporated herein by reference in its entirety.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC.

### INCORPORATION BY REFERENCE OF SEQUENCE LISTING

The Sequence Listing in an XML file, named as 44688_5625_1_SequenceListing of 49,152 bytes, created on Jun. 20, 2025, and submitted to the United States Patent and Trademark Office via Patent Center, is incorporated herein by reference.

### BACKGROUND

RNA plays a critical role in plant cellular activities and phenotypes by serving as messengers (i.e., mRNAs), which facilitate translation of genetic information into proteins, or as modulators (e.g., non-coding RNAs) to regulate gene expression (Großkinsky et al., 2015; Ryu et al., 2019; Yang et al., 2020). Technologies able to capture spatial-temporal dynamics of RNA molecules could be leveraged to unravel the molecular basis of complex phenotypes in plants or facilitate early detection of transcription of plant stress, developmental changes and/or ectopic expression of transgenes. Current technologies for RNA analyses, such as quantitative reverse transcription polymerase chain reaction (qRT-PCR), in situ hybridization and transcriptome-sequencing (Bleckmann and Dresselhaus, 2016; Islam et al., 2024; Martin et al., 2013; Zhao et al., 2021) require destructive, labor-intensive and time-consuming protocols. Recent advancements in biosensors offer an alternative, non-destructive approach for measuring cellular or molecular activities (Liu et al., 2022). For example, RNA labelling technologies using aptamers have been applied for building biosensors for detection of gene expression (Alamos et al., 2021; Bai et al., 2020). However, these technologies require modification of input RNA signals and are thus not feasible for monitoring temporal and spatial patterns of native RNA signals in plants. Therefore, there is a pressing need for innovations in biosensors to enable in vivo detection of RNA molecules within plants.

### SUMMARY

The present disclosure is directed to a split ribozyme biosensor system.

Additionally, a genetically modified plant, plant tissue, or plant cell comprising the split ribozyme biosensor system is described. Methods for examining in vivo RNA expression in plants, plant cells or plant tissues are disclosed. Lastly, disclosed herein is a kit comprising the split ribozyme biosensor system.

In one aspect, the present disclosure is directed to a split ribozyme biosensor system comprising: a first expression cassette comprising a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and a second expression cassette comprising a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein; wherein the first and second fragments of the ribozyme together provide a full-length sequence of the ribozyme, wherein the first and second fragments of the protein together provide a full-length sequence of the protein; and wherein upon transcription in a plant and when a target RNA is present in the plant, the first guide RNA sequence and the second guide RNA sequence bind to the target RNA, and bring the two ribozyme fragments together so that the ribozyme removes itself from flanking sequences to allow formation of an mRNA encoding the full length sequence of the first protein.

In some embodiments, the first protein is a reporter protein. In some embodiments, the first protein is transiently expressed. In some embodiments, the first protein is expressed after the two guide RNAs binding to the target RNA. In some embodiments, the ribozyme comprises an internal guide sequence (IGS) which begins with a guanine and the remainder of the IGS is the reverse complement to the first five base pairs of a P1 helix. In some embodiments, the first fragment of the ribozyme is inserted immediately downstream of an uracil positioned within the nucleotide sequence encoding the first protein such that insertion of the ribozyme creates a fragmentation in the nucleotide sequence encoding the first protein, wherein each fragment of the nucleotide sequence does not encode a functional protein.

In some embodiments, the first polynucleotide sequence further comprises a promoter sequence and a 5′ untranslated sequence that initiates gene expression in the plant and is operably linked to the 5′ of the nucleotide sequence encoding the first fragment of the first protein.

In some embodiments, the first and second polynucleotide sequences are contiguous and under control of the same promoter. In some embodiments, the first and second polynucleotide sequences are under the control of separate promoter sequences and 5′ untranslated sequences.

In some embodiments, the first and second polynucleotide sequence comprises a terminator sequence that signals the RNA polymerase to stop transcription in the plant. In the first polynucleotide sequence, the terminator is operably linked to the 3′ of the first guide RNA sequence, and in the second polynucleotide sequence, the terminator is operably linked to the 3′ of the nucleotide sequence encoding the second fragment of the first protein.

In some embodiments, both the first guide RNA sequence and the second guide RNA sequence range from about 40 to 325 nucleotides in length. In some embodiments, the first RNA guide sequence and/or the second RNA guide sequence each range from about 60 to 170 nucleotides in length. In some embodiments, the first RNA guide sequence and/or the second RNA guide sequence each range from about 80 to 125 nucleotides in length. In some embodiments, the first RNA guide sequence and/or the second RNA guide sequence are each: about 41 nucleotides in length; about 82 nucleotides in length; about 123 nucleotides in length; about 164 nucleotides in length; and/or about 325 nucleotides in length. In some embodiments, the first RNA guide sequence and the second RNA guide sequence are either the same length or different lengths.

In some embodiments, the second polynucleotide further comprises a nucleotide sequence encoding a self-cleaving peptide linked to a second protein, wherein upon translation, the first protein and the second protein are separated. In some embodiments, the second protein is a reporter protein. In some embodiments, the first protein is a reporter protein and the second protein is a reporter protein, and the first and second reporter proteins are different reporter proteins. In some embodiments, the self-cleaving peptide is P2A, E2A, F2A, or T2A.

In some embodiments, the biosensor system further comprises a third polynucleotide sequence comprising a promoter sequence, a 5′ untranslated sequence, a nucleotide sequence encoding a third protein, and a terminator sequence. In some embodiments, the third protein is a reporter protein.

In some embodiments, the reporter protein is a fluorescent protein reporter or bioluminescence reporter protein (luciferase).

Another aspect of the current disclosure is related to methods for examining in vivo RNA expression in plants, plant cells, or plant tissues, the method comprising introducing the biosensor system described herein into a plant, plant cell, or plant tissue to create a genetically modified plant, plant cell, or plant tissue. In some embodiments, the first protein of the biosensor system is a reporter protein, the method further comprising detecting the reporter protein as indicative of the presence of the target RNA. In some embodiments, the second protein of the biosensor system is a reporter protein, the method further comprising detecting the reporter protein as indicative of the presence of the target RNA. In some embodiments, the detecting of the reporter protein occurs through optical imaging or electrochemical imaging. In some embodiments, the optical imaging is selected from fluorescence imaging, bioluminescence imaging, RGB imaging, hyperspectral imaging, and Raman imaging.

In some embodiments, the method further comprises subjecting the genetically modified plant, plant cells, or plant tissue to experimental conditions before the step of detecting the reporter protein, wherein the experimental conditions comprise abiotic stress, biological stress, improved growth conditions.

Another aspect of the current disclosure is directed to a genetically modified plant, plant cell, or plant tissue comprising the biosensor system described herein.

Another aspect of the current disclosure is directed to a kit comprising: a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein. In some embodiments, the kit further comprises a third nucleotide sequence encoding a second protein. In some embodiments, the polynucleotide sequences of the kit can be delivered into plants or plant cells using various methods, including biological methods mediated by bacteria (Agrobacterium/Rhizobium) or viral vectors, physical methods mediated by gene gun or electroporation, and chemical methods mediated polyethylene glycol (PEG), polymers, lipids, or cell-penetrating peptides.

### BRIEF DESCRIPTION OF DRAWINGS

The patent or application file contains at least one drawing executed in color. Copies of this patent or patent application publication with color drawing(s) will be provided by the Patent and Trademark Office upon request and payment of the necessary fee.

FIG. 1a-h. Evaluation of ribozyme splicing activity in Nicotiana benthamiana using sfGFP as the reporter via Agrobacterium-mediated leaf infiltration. (a) Illustration of splicing activity assay using a sfGFP CDS (green) with the Tetrahymena thermophila ribozyme DNA sequence (dark blue) inserted within codon Y66. (b) Construct design for testing ribozyme splicing activity in the presence of the HP14 hairpin structure using sfGFP as the reporter. 35S, Promoter region of the Cauliflower mosaic virus 35S gene; 5′UTR, 5′ untranslated region of Arabidopsis thaliana COLD REGULATED 47 gene; dR, a catalytically dead G264A mutant ribozyme with IGS and P1 loop removed; HSP, terminator region of A. thaliana HEAT SHOCK PROTEIN 18.2 gene; R, ribozyme; sfGFP, super folding GREEN FLUORESCENT PROTEIN. (c) Construct design for testing ribozyme splicing activity without HP14 hairpin structure using sfGFP as the reporter. (d) Evaluation of ribozyme splicing activity with sfGFP as reporter in the presence of HP14 in N. benthamiana. (e) Evaluation of ribozyme splicing activity with sfGFP as reporter in the absence of HP14 in N. benthamiana. (f) Western blot analysis of sfGFP resulting from the reassembly of sfGFP fragments caused by ribozyme-mediated transcript splicing. (g) Bright field of pictures in FIG. 1d. (h) Bright field of pictures in FIG. 1e. Pictures of FIGS. 1g and 1h taken with a Zeiss LSM 710 confocal microscope under bright light. Scale bar=100 μm.

FIG. 2a-f. Split ribozyme-based biosensor system for RNA detection in plants using transient expression. (a) Illustration of the split-ribozyme based biosensor system using a transcript input to produce functional sfGFP. (b) Construct design for evaluation of the function of split ribozyme in plants using red fluorescent protein as transcript input. 35S, Promoter region of the Cauliflower mosaic virus 35S gene: 5′UTR, 5′ untranslated region of Arabidopsis thaliana COLD REGULATED 47 gene; HSP, terminator region of Arabidopsis thaliana HEAT SHOCK PROTEIN 18.2 gene; RFP, RED FLUORESCENT PROTEIN. Green rectangles represent split sfGFP fragments, red rectangles represent split ribozyme fragments, yellow shapes represent two 41-nt gRNAs targeting RFP, pink rectangles represent two 82-nt gRNAs targeting RFP. (c) Split ribozyme-based biosensor system for detecting RFP transcripts in N. benthamiana using sfGFP as reporter via Agrobacterium-mediated leaf infiltration. (d) Characterization of fluorescence intensity for different lengths of gRNAs targeting RFP. The double star indicates P-value less than 0.01. Error bars indicate error bars standard deviation of n=3 biological replicates. (e) Bright field of pictures in FIG. 2c. (f) Florescent signals and bright field of split-ribozyme system targeting RFP transcript with different gRNA length. Pictures of FIGS. 2e and 2f were taken with a Zeiss LSM 710 confocal microscope. Scale bar=100 μm.

FIGS. 3a-f. Utilizing the split ribozyme-based biosensor for imaging different RNA signals in plants. (a) Construct design for detection of various types of RNA signals in plants. 35S. Promoter region of the Cauliflower mosaic virus 35S gene; 5′UTR, 5′ untranslated region of Arabidopsis thaliana COLD REGULATED 47 gene; HSP, terminator region of Arabidopsis thaliana HEAT SHOCK PROTEIN 18.2 gene; blue rectangles represent 82-nt gRNAs targeting Arabidopsis thaliana gene PLT1 (AT3G20840) and purple and brown rectangles represent 82-nt gRNAs targeting different regions of the TRV RNA. (b) Split ribozyme-based biosensor system for detecting the AT3G20840 transcript in N. benthamiana using sfGFP as reporter via Agrobacterium-mediated leaf infiltration. (c) Testing the split ribozyme-based biosensor system for detecting the TRV RNA in N. benthamiana using sfGFP as reporter via Agrobacterium-mediated leaf infiltration. (d) Split ribozyme-based biosensor system for detecting the expression of an endogenous gene (i.e., AT3G20840) in the roots of transgenic A. thaliana plants engineered with the construct pXYB147 using Agrobacterium-mediated stable transformation. (e) Bright field of pictures in FIG. 3b. (f) Bright field of pictures in FIG. 3c. The pictures of FIGS. 3e and 3f were taken with a Zeiss LSM 710 confocal microscope under bright light. Scale bar=100 μm

FIG. 4a-f. Engineering the ribozyme system for interchangeable protein outputs in Nicotiana benthamiana using GFPuv as the reporter via Agrobacterium-mediated leaf infiltration. (a) Construct design for testing the reassembly of GFPuv fragments mediated by ribozyme splicing. GFPuv, enhanced YELLOW GREEN FLUORESCENT LIKE PROTEIN that can be detected by unaided eyes under ultraviolet light. (b) Evidence of the reassembly of GFPuv fragments mediated by ribozyme splicing in N. benthamiana. (c) Construct design for testing the ribozyme splicing activity with sfGFP-P2A-GFPuv fusion protein as the reporter. 35S, Promoter region of the Cauliflower mosaic virus 35S gene; 5′UTR, 5′ untranslated region of Arabidopsis thaliana COLD REGULATED 47 gene; dR, a catalytically dead G264A mutant ribozyme with IGS and P1 loop removed; HSP, terminator region of Arabidopsis thaliana HEAT SHOCK PROTEIN 18.2 gene; P2A, a 2A self-cleaving peptide; R. Ribozyme. (d) Ribozyme-mediated reassembly of sfGFP transcript connected to eYGFP via P2A. (e) Visualization of eYGFPuv, connected via P2A, to sfGFP reassembled via ribozyme splicing in N. benthamiana. (f) Bright field of pictures in FIG. 4d. The pictures were taken with a Zeiss LSM 710 confocal microscope under bright light. Scale bar=100 μm.

FIG. 5. Illustration of the potential applications of the “Plant RNA Vision” technology. The “Plant RNA Vision” toolkit can be used for the detection of gene expression at cellular, tissue/organ and whole-plant levels in response to internal (e.g., hormonal signals, water and nutrient levels, developmental programs) and external stimuli including abiotic (e.g., light, temperature, water, chemical stimuli, mechanical stimuli) and biotic factors (e.g., pathogens, herbivores, beneficial microbes). The biosensor agent includes two plasmid DNA vectors, with each vector containing a partial fragment of a ribozyme, flanked by a partial fragment of a reporter gene (e.g., a gene encoding a fluorescent protein) on one end and a gRNA on the other end. The two gRNAs are designed to specifically base pair with the two neighboring regions, respectively, of an RNA target of interest. The biosensor agent can be delivered into plant cells using various approaches mediated by viruses, agrobacteria, cell penetrating peptides or nanoparticles.

FIG. 6a-b. (a) and (b) Green fluorescent signal observed in the roots of two different transgenic Arabidopsis thaliana plants. The pictures were taken with a Zeiss LSM 710 confocal microscope. Scale bar=100 μm.

FIG. 7a-b. (a) Construct design for testing different RNA stability motifs and new design of Ribozyme with new internal guide sequence (IGS). 35S, Promoter region of the Cauliflower mosaic virus 35S gene: 5′UTR, 5′ untranslated region of Arabidopsis thaliana COLD REGULATED 47 gene; Blue rectangle, tEvopreq motif; Brown rectangle, Triplex motif; R with yellow rectangle: Ribozyme with IGS2; dR with dark red rectangle, a catalytically dead G264A mutant ribozyme with IGS and P1 loop removed; dR with green rectangle, a catalytically dead G264A mutant ribozyme with IGS2; GFPuv, enhanced YELLOW GREEN FLUORESCENT LIKE PROTEIN; HSP, terminator region of A. thaliana HEAT SHOCK PROTEIN 18.2 gene; Pink rectangle, gq2 motif; R with black rectangle, ribozyme. (b) Visualization of eYGFPuv in leaf under UV light, 3 days post N. benthamiana leaf infiltration.

FIG. 8. Illustration of guide RNA design for use in the split ribozyme biosensor system disclosed herein.

FIG. 9. Generic representation of the group I intron structure with emphasis on key loops and domains featuring conserved sequences. Gray regions indicate unpaired segments (loops) that may contain Open Reading Frames (ORFs). Orange rectangles show the location of conserved sequences P. Q. R. S. and IGS. Black rectangles represent exons located in the upstream and downstream regions of the 5′ and 3′ splice sites (green triangles), respectively. The binding site for exogenous guanine (ExoG) on the G-binding site, located in loop P7, is represented by an asterisk.

### DETAILED DESCRIPTION

In one aspect, the present disclosure is directed to a split ribozyme biosensor system. In another aspect, the disclosure is directed to a genetically modified plant, plant tissue, or plant cell comprising the split ribozyme biosensor system described herein. In still another aspect, the disclosure is directed to methods for examining in vivo RNA expression in plants, plant cells or plant tissues. In a further aspect, the disclosure is directed to a kit comprising the split ribozyme biosensor system described herein.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

The terms “genetically modified.” “genetically engineered.” “recombinant cell,” and “recombinant strain” are used interchangeably herein and refer to bacterial cells that have been genetically modified by the cloning and transformation methods of the present disclosure. Thus, the terms include a plant that has been genetically altered, modified, or engineered, such that it exhibits an altered, modified, or different genotype and/or phenotype (e.g., when the genetic modification affects coding nucleic acid sequences of the plant), as compared to the naturally occurring plant from which it was derived. It is understood that the terms refer not only to the particular recombinant plant in question, but also to the progeny or potential progeny of such a plant. Non-viral vectors and transfection reagents, including cationic lipids or non-liposomal lipids, are non-limiting examples of safe tools for introducing exogenous nucleic acids into cells. Transfected nucleic acids are incorporated into the endosome via endocytosis and exposed to the cytosol upon endosome rupture. In some instances, a cell is genetically altered through introducing exogenous nucleic acid without integrating into its genome. This modification is a temporary modification known as a transient transfection, which will not be included in progeny. An example of such genetic alteration occurs with plasmids. Plasmids offer a flexible and reversible way to modify gene expression without making permanent changes to the host genome. Expression levels can be tuned by controlling plasmid copy number or through the activity of plasmid-encoded regulators. Other methods of introducing exogenous nucleic acids into plants are known in the art. Non-limiting examples of such methods include but are not limited to Agroinfiltrations and the floral dip method.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences”, “homologs”, or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Examples of alignment programs include but are not limited to: Mac Vector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “heterologous” refers to a relationship or linkage of two nucleic acid or protein sequences that is not naturally found in a particular organism.

As used herein, the term “exogenous” refers to a substance coming from a source other than its native source. For example, the terms “exogenous protein” and “exogenous gene” refer to a protein and gene that have been artificially supplied to a biological system (e.g., tissue, cell or intracellular site) from a source non-native to the biological system. Artificially mutated variants of endogenous genes are considered “exogenous” for the purposes of this disclosure.

As used herein, “promoter” refers to a DNA sequence capable of controlling the expression of a coding sequence or functional RNA. The promoter sequence may consist of proximal and more distal upstream elements, the latter elements often referred to as enhancers. There are several types of promoters, including constitutive promoters, tissue-specific promoters, inducible promoters, and synthetic promoters. Constitutive promoters are active in most tissues and developmental stages, making them useful for general gene expression. Examples include the CaMV 35S promoter from Cauliflower Mosaic Virus and the maize ubiquitin promoter. Tissue-specific promoters are active only in specific tissues, such as leaves, roots, or seeds. This allows for targeted gene expression, for example, to express a gene only in the seed for increased nutritional value. Inducible promoters are activated by specific stimuli, such as light, hormones, chemicals, or stress. This allows for gene expression to be controlled in response to environmental changes or other factors. Examples include stress-responsive promoters and hormone-responsive promoters. Synthetic promoters are designed and engineered to have specific properties, such as tunable expression levels or specific tissue targeting, and can be combinations of elements from different promoters.

The term “operably linked” means in this context the sequential arrangement of the promoter polynucleotide with a further oligo- or polynucleotide, resulting in transcription of the further polynucleotide. In some embodiments, the promoter sequences are located just prior to a gene's 5′UTR, or open reading frame.

As used herein, a “system” refers to a combination of multiple components or products which can interact in a way to produce a desired result. In some embodiments, the components could be provided in the form of a kit. In some embodiments, the disclosure provides kits containing any one or more of the elements disclosed in the above methods and compositions. In some embodiments, the kit comprises a vector system and instructions for using the kit.

### Split Ribozyme Biosensor System

The split ribozyme biosensor system of the disclosure converts transcriptional signals into orthogonal protein outputs in plants, plant cells, and plant tissues through the use of fragmented self-splicing ribozymes and fragmented protein genes.

As used herein, “fragmented ribozyme” refers to a ribozyme that has been split into two fragments, where each fragment is a portion of the nucleic acid sequence of the ribozyme that lacks the catalytic function of the full-length ribozyme. The location of the split in the ribozyme is not limited to a particular site, as long as each of the two fragments resulting from the split lacks the catalytic function of the full-length ribozyme. Likewise, as used herein, a fragmented protein gene (i.e., a gene encoding a protein) refers to a gene that has been split into two fragments, where each fragment is a portion of the full-length gene and encodes a polypeptide that lacks the function of the full-length protein encoded by the full-length gene. The location of the split in the gene is not limited to a particular site, as long as each of the two gene fragments resulting from the split does not encode a polypeptide that has the function of the full-length protein. As such, the split ribozyme biosensor system of the disclosure uses ribozyme and gene fragments so that a functional transcript encoding the full-length protein can only be made when the ribozyme excises itself from the surrounding exons and joins the split segments of the gene transcripts together in a manner that is specific to the presence of a target mRNA.

For example, a DNA sequence encoding the Tetrahymena ribozyme is split into two fragments while a gene encoding a reporter protein is split into two fragments. One ribozyme fragment is fused to one of the reporter protein gene fragments on one end and a first guide RNA (gRNA) on the other end, while the other ribozyme fragment is fused to the other reporter protein gene fragment on one end and a second gRNA on the other. The two gRNAs, which are attached to the two partial ribozyme fragments, are designed to specifically base pair with the two neighboring regions, respectively, of a target RNA of interest. Upon DNA-to-mRNA transcription, the two ribozyme fragments are brought together by the binding of gRNAs to the target RNA of interest if present, resulting in the combination of the ribozyme fragments allowing the self-splicing of the ribozyme and consequently, the reassembly of the two mRNA fragments of the reporter protein gene into a full-length transcript, which can be converted to a visible output (e.g., a fluorescent protein) via mRNA-to-protein translation. The split ribozyme reassembly and subsequent splicing occur exclusively in the presence of the target RNA of interest, ensuring specificity of the reporter protein.

Ribozymes (ribonucleic acid enzymes) are RNA molecules that have the ability to catalyze specific biochemical reactions, including RNA splicing in gene expression, similar to the action of protein enzymes. The most common activities of natural or in vitro evolved ribozymes are the cleavage (or ligation) of RNA and DNA and peptide bond formation. Ribozymes function within the ribosome as part of the large subunit ribosomal RNA to link amino acids during protein synthesis while also participating in a variety of RNA processing reactions, including RNA splicing, viral replication, and transfer RNA biosynthesis. The split ribozyme systems of the disclosure use a self-splicing ribozyme to splice the RNA. RNA splicing is a process of pre-mRNA maturation prior to translation, consisting of two transesterification reactions. As a result, the intron is cut out and the two remaining exons are ligated. This process is mediated in eukaryotes mainly by the spliceosome, an RNA-protein complex. The spliceosome does not exist in prokaryotes and archaea. Instead, RNA splicing in prokaryotes and archaea is mediated by the action of self-splicing group I or II introns. Hence, the group I and group II self-splicing introns are ribozymes that, in a similar way to the spliceosome, catalyze two transesterification reactions. Group II introns and the spliceosome share common architectural features and a common splicing mechanism.

Self-splicing or self-catalyzing ribozymes are RNA molecules that can facilitate their own excision from a larger RNA molecule. Self-splicing ribozymes are known in the art and are found within eukaryotes, prokaryotes, and viruses. Group I and II introns perform splicing similar to the spliceosome without requiring any protein. Group I introns are large self-splicing ribozymes that catalyze their own excision from mRNA, tRNA, and rRNA precursors through two sequential phosphotransesterification reactions. Group I Introns occur in all life domains. In bacteria, they are primarily found in rRNA and tRNA genes but are less common in coding genes. In some protists, plants, and fungi, these introns can be found in nuclear rRNA genes as well as in rRNA, tRNA, and protein-coding genes of organelles, such as fungal mitochondria and plant mitochondria and chloroplasts. Group II introns are a large class of self-catalytic (self-cleaving) ribozymes and mobile genetic elements.

Self-splicing ribozymes for use with the biosensor systems disclosed herein can be derived from group I and group II introns as well as small self-splicing ribozymes. Examples of ribozymes include phage Twort group I ribozyme, hammerhead, hairpin, VS ribozymes, Azoarcus group I intron, and Tetrahymena group I intron. The group I intron Tetrahymena thermopila ribozyme was the first RNA molecule with enzymatic activity that did not involve protein, i.e., ribozyme. As such, one of the self-splicing ribozymes used for the biosensor system of the current disclosure is a Tetrahymena ribozyme. One of ordinary skill in the art will understand that any self-cleaving ribozyme will work in the disclosed biosensor system as long as the function of the ribozyme remains, i.e., catalyzing its own excision from a larger RNA molecule to combine (bring together) two fragments of a protein gene transcript allowing for translation of the protein.

In some embodiments, the self-splicing ribozyme is the Tetrahymena group I ribozyme, which comprises a nucleic acid sequence of SEQ ID NO: 6. In some embodiments, the ribozyme comprises a nucleic acid sequence having at least 90% sequence identity to the nucleic acid sequence SEQ ID NO: 6. In some embodiments, the ribozyme comprises a nucleic acid sequence having at least 91% sequence identity to the nucleic acid sequence SEQ ID NO: 6. In some embodiments, the ribozyme comprises a nucleic acid sequence having at least 92% sequence identity to the nucleic acid sequence SEQ ID NO: 6. In some embodiments, the ribozyme comprises a nucleic acid sequence having at least 93% sequence identity to the nucleic acid sequence SEQ ID NO: 6. In some embodiments, the ribozyme comprises a nucleic acid sequence having at least 94% sequence identity to the nucleic acid sequence SEQ ID NO: 6. In some embodiments, the ribozyme comprises a nucleic acid sequence having at least 95% sequence identity to the nucleic acid sequence SEQ ID NO: 6. In some embodiments, the ribozyme comprises a nucleic acid sequence having at least 96% sequence identity to the nucleic acid sequence SEQ ID NO: 6. In some embodiments, the ribozyme comprises a nucleic acid sequence having at least 97% sequence identity to the nucleic acid sequence SEQ ID NO: 6. In some embodiments, the ribozyme comprises a nucleic acid sequence having at least 98% sequence identity to the nucleic acid sequence SEQ ID NO: 6. In some embodiments, the ribozyme comprises a nucleic acid sequence having at least 99% sequence identity to the nucleic acid sequence SEQ ID NO: 6.

SEQ ID NO: 6:
AAAAGTTATCAGGCATGCACCTGGTAGCTAGTCTTTAAACCAATAGATTG
CATCGGTTTAAAAGGCAAGACCGTCAAATTGCGGGAAAGGGGTCAACAGC
CGTTCAGTACCAAGTCTCAGGGGAAACTTTGAGATGGCCTTGCAAAGGGT
ATGGTAATAAGCTGACGGACATGGTCCTAACCACGCAGCCAAGTCCTAAG
TCAACAGATCTTCTGTTGATATGGATGCAGTTCACAGACTAAATGTCGGT
CGGGGAAGATGTATTCTTCTCATAAGATATAGTCGGACCTCTCCTTAATG
GGAGCTAGCGGATGAAGTGATGCAACACTGGAGCCGCTGGGAACTAATTT
GTATGCGAAAGTATATTGATTAGTTTTGGAGTACTCG.

To enable biosensing, DNA sequence encoding the Tetrahymena ribozyme is split into two inactive fragments, with each fused to a partial fragment of a reporter gene (e.g., a gene encoding a fluorescent protein) on one end and a guide RNA (gRNA) on the other end. The two gRNAs, which are attached to the two partial ribozyme fragments, are designed to specifically base pair with the two neighboring regions, respectively, of an RNA target of interest. Upon DNA-to-mRNA transcription, the two partial ribozyme fragments are brought together by the binding of gRNAs to the RNA target, resulting in the self-splicing of the ribozyme fragments and consequently the reassembly of the two partial mRNA fragments of the reporter gene into a full-length transcript, which can be converted to a visible output (e.g., a fluorescent protein) via mRNA-to-protein translation. The split ribozyme reassembly and subsequent splicing occur exclusively in the presence of a target RNA, ensuring specificity in detection. Moreover, because the system is linked to an orthogonal output, a single binding event between the target RNA and the split ribozyme system can initiate the translation of various functional proteins, acting as an RNA signal transducer.

In one aspect, the present disclosure is directed to a split ribozyme biosensor system comprising: a first expression cassette comprising a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and a second expression cassette comprising a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein; wherein the first and second fragments of the ribozyme together provide a full-length sequence of the ribozyme, wherein the first and second fragments of the protein together provide a full-length sequence of the protein; and wherein upon transcription in a plant and when a target RNA is present in the plant, the first guide RNA sequence and the second guide RNA sequence bind to the target RNA, and bring the two ribozyme fragments together so that the ribozyme removes itself from flanking sequences to allow formation of an mRNA encoding the full length sequence of the first protein.

As used herein, the term “expression cassette” refers to a DNA comprising a gene and a regulatory sequence (such as a transcriptionally regulatory sequence, e.g., a promoter). In a successful transformation of the expression cassette into a plant cell, the expression cassette directs the cellular machinery of the plant cell to make RNA and/or protein.

In some embodiments, the first expression cassette comprises a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence.

In some embodiments, the first protein is a reporter protein. Reporter proteins are well-known in the art and include, but are not limited to, visual reporter proteins. Some commonly known visual reporter proteins in the art are fluorescent proteins. Fluorescent proteins are bioluminescent proteins used as labeling markers in organisms owing to their energy transfer reactions that produce fluorescence without species restrictions or substrate requirements. As potential biomarkers, combined with high-resolution microscopy, fluorescent proteins are widely utilized in the study of various biological processes. Many fluorescent proteins are known in the art including green fluorescent protein (GFP) and its variants (eYFP, mCherry), the GFP-like protein (eYGFPuv), blue fluorescent protein (BFP), cyan fluorescent protein (CFP), yellow fluorescent protein (YFP), red fluorescent protein (RFP), DsRed, betalain-producing RUBY proteins (mRuby2), G3GFP, tdTomato, SfGFP, Clover, YPet, mVenus, mCerulean, and ECFP. Additionally, anthocyanin production, induced by transcription factors like HbAN1 and HbAN2, can be used as a visual marker.

In some embodiments, the reporter protein is sfGFPn Y66. sfGFPn is a, which is encoded by a gene comprising a nucleic acid sequence of SEQ ID NO: 1. In some embodiments, the reporter protein is encoded by a gene comprising a nucleic acid sequence having at least 90% sequence identity to the nucleic acid sequence of SEQ ID NO: 1.

SEQ ID NO: 1:
ATGGTGTTCAATGCTTTTCCCGTTATCCGGATCACATGAAACGGCATGAC
TTTTTCAAGAGTGCCATGCCCGAAGGTTATGTACAGGAACGCACTATATC
TTTCAAAGATGACGGGACCTACAAGACGCGTGCTGAAGTCAAGTTTGAAG
GTGATACCCTTGTTAATCGTATCGAGTTAAAGGGTATTGATTTTAAAGAA
GATGGAAACATTCTTGGACACAAACTCGAGTACAACTTTAACTCACACAA
TGTATACATCACGGCAGACAAACAAAAGAATGGAATCAAAGCTAACTTCA
AAATTCGCCACAACGTTGAAGATGGTTCCGTTCAACTAGCAGACCATTAT
CAACAAAATACTCCAATTGGCGATGGCCCTGTCCTTTTACCAGACAACCA
TTACCTGTCGACACAATCTGTCCTTTCGAAAGATCCCAACGAAAAGCGTG
ACCACATGGTCCTTCTTGAGTTTGTAACTGCTGCTGGGATTACACATGGC
ATGGATGAGCTCTACAAATAA.

In some embodiments, the reporter protein is sfGFPn, which is encoded by a gene comprising a nucleic acid sequence of SEQ ID NO: 2. In some embodiments, the reporter protein is encoded by a gene comprising a nucleic acid sequence having at least 90% sequence identity to the nucleic acid sequence of SEQ ID NO: 2.

SEQ ID NO: 2:
ATGAGCAAAGGAGAAGAACTTTTCACTGGAGTTGTCCCAATTCTTGTTGA
ATTAGATGGTGATGTTAATGGGCACAAATTTTCTGTCCGTGGAGAGGGTG
AAGGTGATGCTACAAACGGAAAACTCACCCTTAAATTTATTTGCACTACT
GGAAAACTACCTGTTCCGTGGCCAACACTTGTCACTACTCTGACCT

In some embodiments, the reporter protein is eYGFPuv, which is encoded by a gene comprising a nucleic acid sequence of SEQ ID NO: 3. In some embodiments, the reporter protein is encoded by a gene comprising a nucleic acid sequence having at least 90% sequence identity to the nucleic acid sequence of SEQ ID NO: 3.

SEQ ID NO: 3:
ATGACAACCTTCAAAATCGAGTCCCGGATCCACGGCAACCTCAACGGGGA
GAAGTTCGAGTTGGTTGGAGGTGGAGTAGGTGAGGAGGGTCGCCTCGAGA
TTGAGATGAAGACTAAAGATAAACCACTGGCATTCTCTCCCTTCCTGCTG
ACCACTTGCATGGGTTACGGGTTCTACCACTTCGCCAGCTTCCCAAAGGG
GATTAAGAACATCTATCTTCATGCTGCAACGAACGGAGGTTACACCAACA
CCAGGAAGGAGATCTATGAAGACGGCGGCATCTTGGAGGTCAACTTCCGT
TACACTTACGAGTTCAACAAGATCATCGGTGACGTCGAGTGCATTGGACA
TGGATTCCCAAGTCAGAGTCCGATCTTCAAGGACACGATCGTGAAGAGTT
GTCCCACGGTGGACCTGATGTTGCCAATGTCCGGGAACATCATCGCCAGC
TCCTACGCTTACGCCTTCCAACTGAAGGACGGCTCTTTCTACACGGCAGA
AGTCAAGAACAACATAGACTTCAAGAATCCAATCCACGAGTCCTTCTCGA
AGTCAGGGCCCATGTTCACCCACAGACGTGTCGAGGAGACTCTCACCAAG
GAGAACCTTGCCATAGTGGAGTACCAGCAGGTTTTCAACAGCGCCCCAAG
AGACATGTAG.

In some embodiments, the reporter protein is Red Fluorescent Protein (RFP), which is encoded by a gene comprising a nucleic acid sequence of SEQ ID NO: 4. In some embodiments, the reporter protein is encoded by a gene comprising a nucleic acid sequence having at least 90% sequence identity to the nucleic acid sequence of SEQ ID NO: 4.

SEQ ID NO: 4:
ATGGCGAGTAGCGAAGACGTTATCAAAGAGTTCATGCGTTTCAAAGTTCG
TATGGAAGGTTCCGTTAACGGTCACGAGTTCGAAATCGAAGGTGAAGGTG
AAGGTCGTCCGTACGAAGGTACCCAGACCGCTAAACTGAAAGTTACCAAA
GGTGGTCCGCTGCCGTTCGCTTGGGACATCCTGTCCCCGCAGTTCCAGTA
CGGTTCCAAAGCTTACGTTAAACACCCGGCTGACATCCCGGACTACCTGA
AACTGTCCTTCCCGGAAGGTTTCAAATGGGAACGTGTTATGAACTTCGAA
GACGGTGGTGTTGTTACCGTTACCCAGGACTCCTCCCTGCAAGACGGTGA
GTTCATCTACAAAGTTAAACTGCGTGGTACCAACTTCCCGTCCGACGGTC
CGGTTATGCAGAAAAAAACCATGGGTTGGGAAGCTTCCACCGAACGTATG
TACCCGGAAGACGGTGCTCTGAAAGGTGAAATCAAAATGCGTCTGAAACT
GAAAGACGGTGGTCACTACGACGCTGAAGTTAAAACCACCTACATGGCTA
AAAAACCGGTTCAGCTGCCGGGTGCTTACAAAACCGACATCAAACTGGAC
ATCACCTCCCACAACGAAGACTACACCATCGTTGAACAGTACGAACGTGC
TGAAGGTCGTCACTCCACCGGTGCTTAA.

In some embodiments, the first protein is transiently expressed. In some embodiments, the first protein is constitutively expressed. In some embodiments, the first protein is expressed after the two guide RNAs bind to the target RNA. The two guide RNAs when bound to the target RNA bring the ribozyme fragments into proximity of each other so the ribozyme fragments can combine to form the complete ribozyme. The ribozyme then self-splices from the combination allowing the two fragments of the first protein to combine and be expressed.

The self-splicing of the ribozyme occurs through two sequential phosphotransesterification reactions catalyzed by group I introns. First, a free guanosine is bound to the ribozyme and its 3′ hydroxyl group is positioned to attack the phosphorous atom at the 5′ splice site. The guanosine becomes covalently attached to the 5′ terminus of the intron. Second, the phosphodiester bond located at the 3′ splice site undergoes attack from the newly freed 3′ hydroxyl group of the 5′ exon, giving rise to ligated exon sequences.

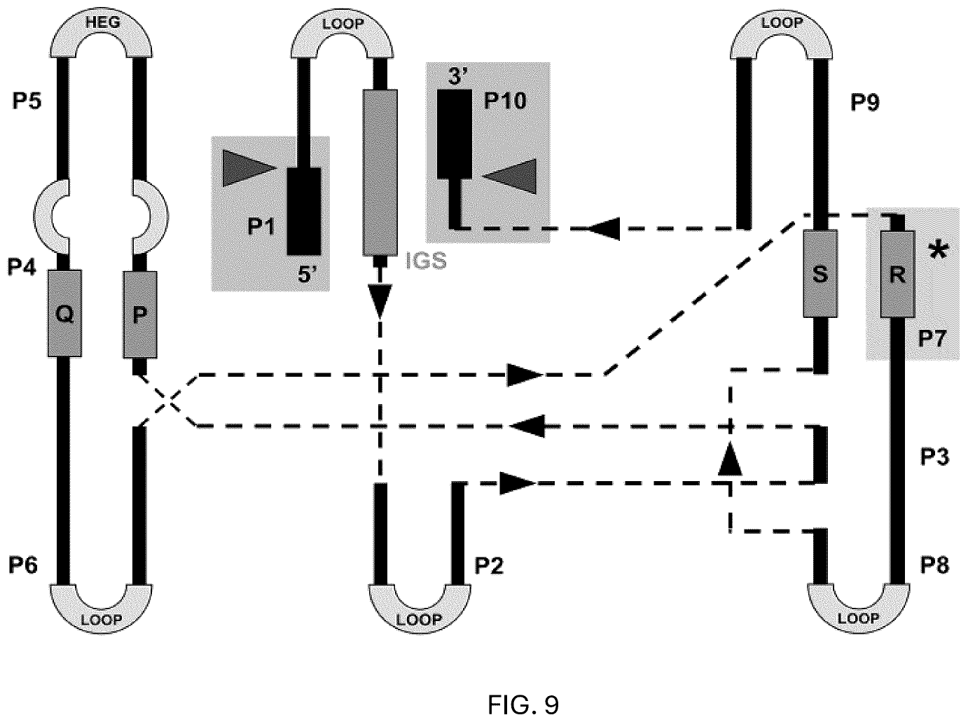

Group I introns have been found to comprise a catalytic core, located at the junction of the P4-P6 (P4, P5, P6) and P3-P9 (P3, P7, P8, P9) domains, formed by intramolecular base pairing (FIG. 9). P1 and P10 define the 5′ and 3′ splicing sites, respectively, which are formed by base pairing between an internal guide sequence (IGS) located immediately upstream of the 5′ splicing site and an exon sequence adjacent to the splicing site. Therefore, ribozyme-mediated cleavage at the 5′ splice site depends on formation of the base-paired helix P1 with the IGS and sequences adjacent the splice site. Additionally, the presence of a U.G “wobble” base-pair (a non-Watson-Crick base pairing interaction in RNA, where a guanine (G) base pairs with a uracil (U) base instead of the usual cytosine (C) or adenine (A)) positioned four, five or six residues from the base of this helix defines the susceptible phosphodiester bond. The final nucleotide of the intron, called @G, and the first nucleotide of the 3′ exon define the 3′ splicing site, and the interaction between the adjacent portion of the IGS and the first nucleotide of the 3′ exon constitutes P10.

During ribozyme excision, the nucleotides of the IGS base pair with nucleotides of P1, forming the P1 loop (see FIG. 9). Through specific tertiary interactions between the P1 loop and the ribozyme core, the P1 loop is docked into the ribozyme active site.

In some embodiments, the nucleotide sequence encoding the first fragment of the ribozyme is inserted immediately downstream of an uracil where the uracil is located in the nucleotide sequence encoding the first protein. Therefore, in some embodiments, the first fragment of the ribozyme is inserted immediately downstream of an uracil where the IGS starts with a guanine and the remaining IGS sequence is in reverse complementation to the first 5 base pairs of the P1 helix, allowing creation of the P1 loop. The uracil is positioned within the nucleotide sequence encoding the first protein such that insertion of the ribozyme creates the fragmentation in the nucleotide sequence encoding the first protein, creating the first fragment and second fragment of the nucleotide sequence encoding the first protein. The fragmentation of the nucleotide sequence encoding the first protein into a first fragment and a second fragment of the nucleotide sequence is such that each fragment does not encode a functional protein, i.e. the first fragment of the nucleotide sequence does not encode a functional protein and the second fragment of the nucleotide sequence does not encode a functional protein.

In some embodiments, the ribozyme comprises an internal guide sequence (IGS) which begins with a guanine and the remainder of the IGS is the reverse complement to the first five base pairs of a P1 helix (creating the P1 loop, see FIG. 9). An IGS is a short sequence within an intron that base pairs with sequences in the flanking exons, allowing for the splicing of group I introns. This interaction is essential for bringing the exons into the correct alignment for ligation during the self-splicing process.

In some embodiments, the P1 loop comprises a nucleic acid sequence of AAATAGCAATATTTACCTTT (SEQ ID NO: 10). In some embodiments, the split site of the ribozyme occurs in the P1 loop where the P1 loop is split into a fragment and the first fragment comprises a nucleic acid sequence of AAATAGCAA while the second fragment of the P1 loop comprises a nucleic acid sequence of TATTTACCTTT (SEQ ID NO: 11).

In some embodiments, the split site occurs in the IGS of the ribozyme so that a first fragment of the ribozyme comprises a first fragment of the IGS and a second fragment of the ribozyme comprises a second fragment of the IGS. In some embodiments, the IGS of the first fragment of the ribozyme comprises a nucleic acid sequence of GGGTCA. In some embodiments, the IGS of the second fragment of the ribozyme comprises a nucleic acid sequence of GTAGAT.

In some embodiments, the first polynucleotide sequence further comprises a promoter sequence and a 5′ untranslated (UTR) sequence that initiates gene expression in the plant and is operably linked to the 5′ of the nucleotide sequence encoding the first fragment of the first protein.

In some embodiments, the promoter sequence is a nucleic acid sequence in a polynucleotide that proteins bind to for the initiation of transcription of a single RNA transcript from the nucleic acids downstream of the promoter. For the described invention, any promoter which is functional in plants will work. Promoters that function in plants are known in the art. Some non-limiting examples of common promoters used in plants include CaMV35S promoter; ubiquitin (Ubi) promoters such as Ubi-1 from maize, RUBQ1/RUBQ2 from rice, Gmubi, and Ubi from Arabidopsis; Actin promoters, Alcohol dehydrogenase (Adh) reporters; sugarcane bacilliform virus reporters; Nopaline synthase gene reporter; cestrum yellow leaf curling virus (CmYLCV); P OsCon1; KST1; TCTP; heat shock protein reporters (HSP); engineered reporters; and various other promoters identified from plant viruses and endogenous genes, including those from the figwort mosaic virus, mirabilis mosaic virus, and others. While promoters known to work in plants are known in the art, one of skill in the art will understand that certain promoters will work better than others and certain promoters will work better in dicots while other promoters will work better in monocots.

In some embodiments, the promoter sequence is that of a 35S promoter region of the Cauliflower mosaic virus (CaMV35S). In some embodiments, the promoter sequence of a 35S promoter comprises a nucleic acid sequence of SEQ ID NO: 5. In some embodiments, the promoter sequence comprises a nucleic acid sequence having at least 90% sequence identity to the nucleic acid sequence SEQ ID NO: 5. One of skill in the art will understand that the biological function of the promoter will remain even with the variation in the sequence, i.e., bind proteins for the initiation of transcription of a single RNA transcript from the nucleic acids downstream of the promoter sequence.

SEQ ID NO: 5:
TGAGACTTTTCAACAAAGGGTAATATCCGGAAACCTCCTCGGATTCCATT
GCCCAGCTATCTGTCACTTTATTGTGAAGATAGTGGAAAAGGAAGGTGGC
TCCTACAAATGCCATCATTGCGATAAAGGAAAGGCCATCGTTGAAGATGC
CTCTGCCGACAGTGGTCCCAAAGATGGACCCCCACCCACGAGGAGCATCG
TGGAAAAAGAAGACGTTCCAACCACGTCTTCAAAGCAAGTGGATTGATGT
GATATCTCCACTGACGTAAGGGATGACGCACAATCCCACTATCCTTCGCA
AGACCCTTCCTCTATATAAGGAAGTTCATTTCATTTGGAGAGAACA.

In some embodiments, the first polynucleotide sequence comprises a 5′ UTR. The 5′ UTR is the region of a messenger RNA (mRNA) located directly upstream from the initiation codon. The 5′ UTR is important for the regulation of translation. 5′ UTRs that function in plants are known in the art. Any 5′ UTR known to function in plants will function in the biosensor system disclosed herein. Some non-limiting examples of 5′UTRs known to function in plants include 5′ UTRs from Arabidopsis thaliana and Nicotiana benthamiana. Some specific examples of 5′ UTRs include GGT1 and GGT2, Alfalfa mosaic virus RNA 1, psbA, cold-regulated 47 gene, and the synthetic 5′ UTR (synJ).

In some embodiments, the 5′ UTR sequence is a 5′ UTR of an Arabidopsis thaliana cold regulated 47 gene. In some embodiments, the 5′ UTR of an Arabidopsis thaliana cold regulated 47 gene comprises a nucleic acid sequence of SEQ ID NO: 27. In some embodiments, the 5′ UTR sequence comprises a nucleic acid sequence having at least 90% sequence identity to the nucleic acid sequence SEQ ID NO: 27. One of skill in the art will understand that the biological function of the 5′ UTR will remain even with the variation in the sequence, i.e., regulation of translation.

SEQ ID NO: 27:
CAAACATTACTCATTCACAAAACCATCTTAAAGCAACTACACAAGTCTTG
AAATTTTCTCATATTTTCTATTTACTATATAAACTTTTAATCAAATCAAG
ATTAAAG.

In some embodiments, the two (i.e., the first and second) polynucleotide sequences are located on the same vector and under control of the same promoter. In some embodiments, the first and second polynucleotide sequences are under the control of separate promoter sequences and 5′ untranslated sequences.

A terminator sequence is a nucleic acid sequence in a polynucleotide that signals the end of transcription. Any terminator which is functional in plants is available to use in the system disclosed herein. Plant terminators are known to comprise four main cis-elements: the far upstream element (FUE), the near upstream element (NUE), the cleavage site (CS), and the cleavage element (CE). The FUE is a U-rich region that can span 60-130 nucleotides and is located 30 nucleotides or more upstream of the cleavage site which is involved with the efficiency of mRNA 3′ end processing. The NUE is the plant terminator element containing the polyadenylation signal, is located 13-30 nucleotides upstream of the cleavage signal, and is characterized by being A-rich. The CE includes the CS embedded within two short U-rich regions located immediately up- and downstream of the cleavage site. Terminators are located downstream of the gene being transcribed and function as a signal to stop transcription. Terminators that function in plants are known in the art. Some non-limiting examples of common terminators used in plants include: CaMV 35S; nopaline synthase (nos) from Agrobacterium tumefaciens; octopine synthase (ocs) from Agrobacterium tumefaciens; Flaveria bidentis Mel (NADP-malic enzyme); the 3′ regulatory region of the Rubisco small subunit (RBCSIA) from Arabidopsis; the GLUTELIN B-1 (Glub-1) terminator from rice, and heat shock protein terminators (HSP). While terminators known to work in plants are known in the art, one of skill in the art will understand that certain terminators will work better than others and certain terminators will work better in dicots while other terminators will work better in monocots.

In some embodiments, the terminator sequence is a Heat Shock Protein (HSP) sequence. HSP terminator sequences are known in the art and commonly used in genetically modified plants. HSP terminator sequences can be plant specific, i.e. the HSP terminator from Arabodopsis thaliana. In some embodiments, the HSP terminator sequence comprises a nucleic acid sequence of that of SEQ ID NO: 26. In some embodiments, the HSP terminator sequence comprises a nucleic acid sequence having at least 90% sequence identity to the nucleic acid sequence SEQ ID NO: 26. One of skill in the art will understand that the biological function of the terminator sequence will remain even with the variation in the sequence, i.e., termination of translation.

SEQ ID NO: 26:
ATATGAAGATGAAGATGAAATATTTGGTGTGTCAAATAAAAAGCTTGTGT
GCTTAAGTTTGTGTTTTTTTCTTGGCTTGTTGTGTTATGAATTTGTGGCT
TTTTCTAATATTAAATGAATGTAAGATCTCATTATAATGAATAAACAAAT
GTTTCTATAATCCATTGTGAATGTTTTGTTGGATCTCTTCTGCAGCATAT
AACTACTGTATGTGCTATGGTATGGACTATGGAATATGATTAAAGATAAG
ATGGGCTCATAGAGTAAAACGAGGCGAGGGACCTATAAACCTCCCTTCAT
CATGCTATTTCATGATCTATTTTATAAAATAAAGATGTAGAAAAAAGTAA
GCGTAATAACCGCAAAACAAATGATTTAAAACATGGCACATAATGAGGAG
ATTAAGTTCGGTTTACGTTTATTTTAGTACTAATTGTAACGTGAGACTAC
GTATCGGGAATCGCCTAATTAAAGCATTAATGCGAACCTGATTAGATTCA
CCGACCCTCCTATCGTGTCGACCTTTCTGTTTCTTAGAATTTTTTGGTAG
TCTATGTACTAATAATGTCAGCTTCGTATTTATTTCATAAGCAATTTGCA
TTTGCAATTTGTTTTTTACTTTTATTTTTATTGTATTGTGGAATGTGGAC
TCGTACCAACATGAAGTTATATACCACCAAAAAAATTACAGTTAGTCAAA
AGATTCACGAGTGAGAGCTACTTATGATTGTCTTTTACGTATATGTCTAA
TTGTCTATTTGCTCAATAATCTTTGTACTTTCTTTTGTCGTTGATAAAAT
CACAAAGTTCCAAAAGTAATCGAATGATTTGCTTTTAAGAAAAGAAGAGC
TCAATAATTCAACATATATCTGTACACA.

In some embodiments, the first and second polynucleotide sequence each comprises a terminator sequence that signals the RNA polymerase to stop transcription in the plant. In the first polynucleotide sequence, a terminator is operably linked to the 3′ of the first guide RNA sequence, and in the second polynucleotide sequence, a terminator is operably linked to the 3′ of the nucleotide sequence encoding the second fragment of the first protein. The terminators linked to the first and second polynucleotide sequences can have the same sequence, or different sequences. In some embodiments, where the first and second polynucleotides are located on the same vector and under control of the same promoter, the first and second polynucleotides share one terminator sequences. In some embodiments, where the first and second polynucleotides are under the control of separate promoters, the first and second polynucleotides have separate terminator sequences.

The guide RNA (gRNA) of the split ribozyme biosensor system described herein function to base pair with the RNA target of interest. The two gRNAs, which are attached to the two partial ribozyme fragments, are designed to specifically for the RNA target of interest. Upon DNA-to-mRNA transcription, the two ribozyme fragments are brought together by the binding of gRNAs to the RNA target of interest. This results in the combination of the ribozyme fragments allowing the self-splicing of the ribozyme and consequently, the reassembly of the two mRNA fragments of the reporter protein.

In some embodiments, the first guide RNA sequence ranges from about 40 to 325 nucleotides in length. In some embodiments, the first guide RNA sequence ranges from about 60 to 170 nucleotides in length. In some embodiments, the first guide RNA sequence ranges from about 80 to 125 nucleotides in length. In some embodiments, the first guide RNA sequence is about 41 nucleotides in length. In some embodiments, the first guide RNA sequence is about 82 nucleotides in length. In some embodiments, the first guide RNA sequence is about 123 nucleotides in length. In some embodiments, the first guide RNA sequence is about 164 nucleotides in length. In some embodiments, the first guide RNA sequence is about 325 nucleotides in length.

In some embodiments, the second guide RNA sequence ranges from about 40 to 325 nucleotides in length. In some embodiments, the second guide RNA sequence ranges from about 60 to 170 nucleotides in length. In some embodiments, the second guide RNA sequence ranges from about 80 to 125 nucleotides in length. In some embodiments, the second guide RNA sequence is about 41 nucleotides in length. In some embodiments, the second guide RNA sequence is about 82 nucleotides in length. In some embodiments, the second guide RNA sequence is about 123 nucleotides in length. In some embodiments, the second guide RNA sequence is about 164 nucleotides in length. In some embodiments, the second guide RNA sequence is about 325 nucleotides in length.

In some embodiments, the first RNA guide sequence and the second RNA guide sequence are the same length. In some embodiments, the first guide RNA sequence and the second guide RNA sequence are different lengths.

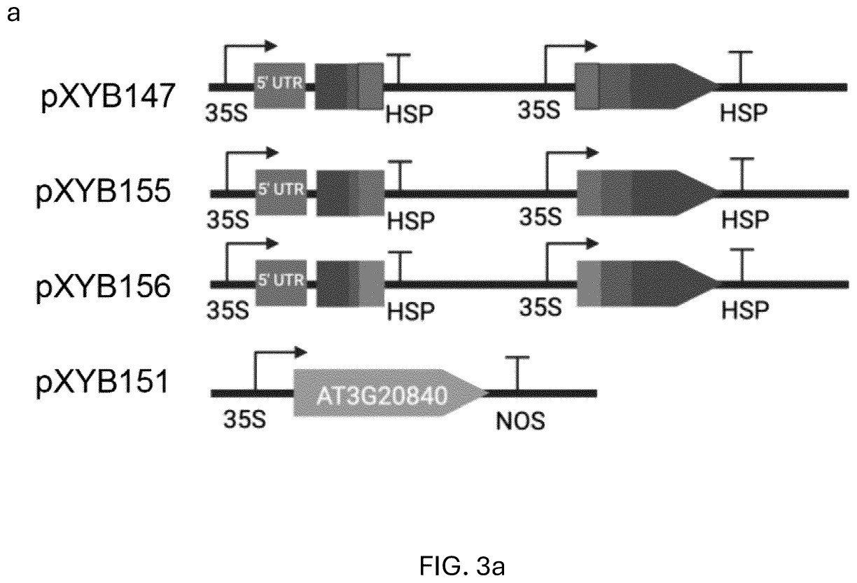

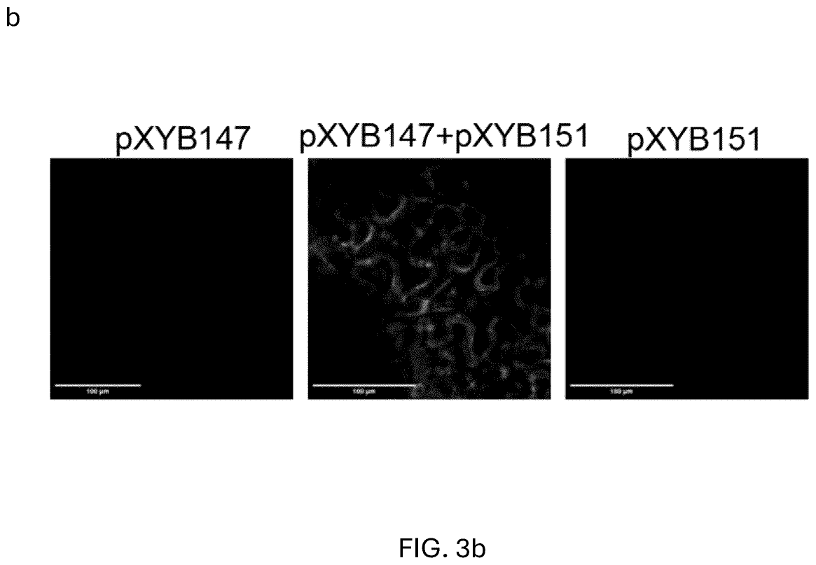

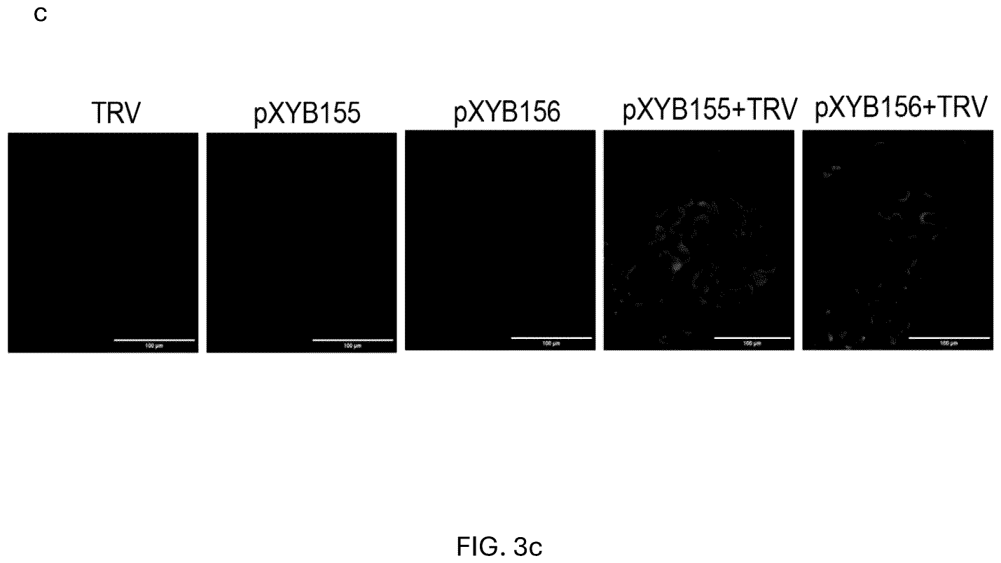

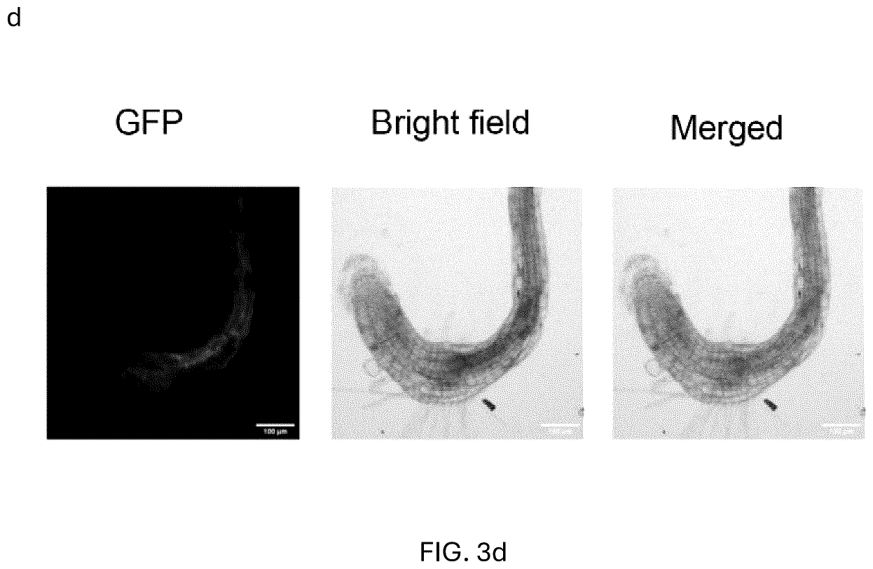

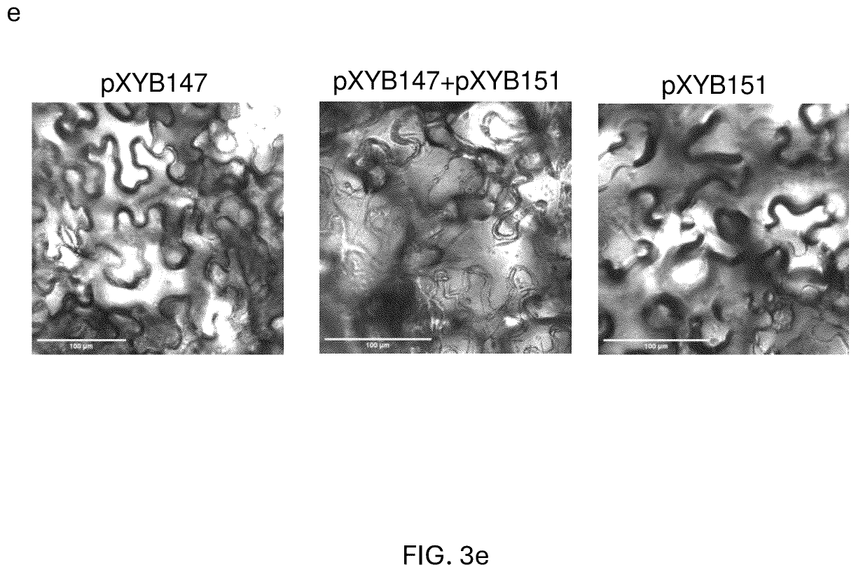

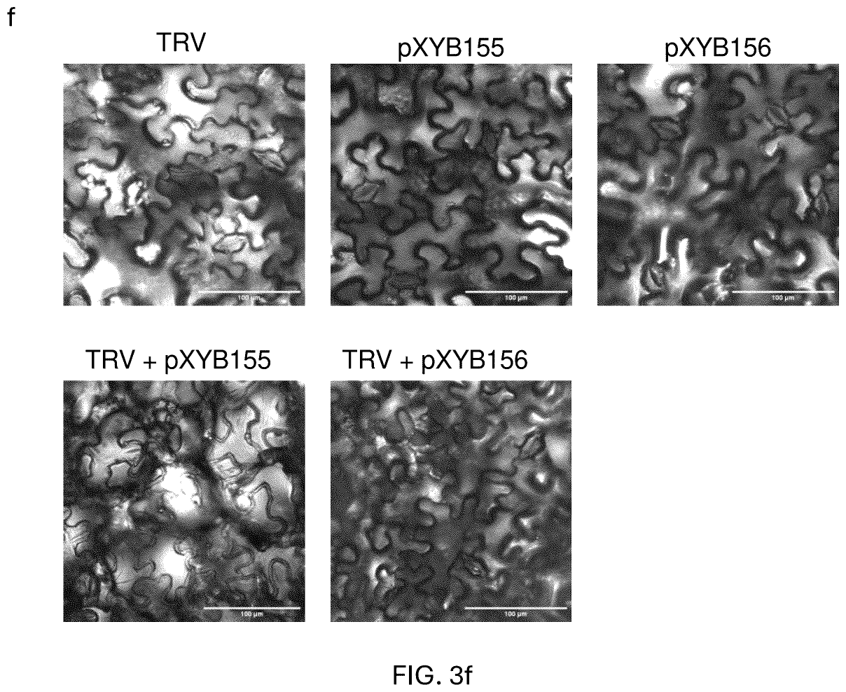

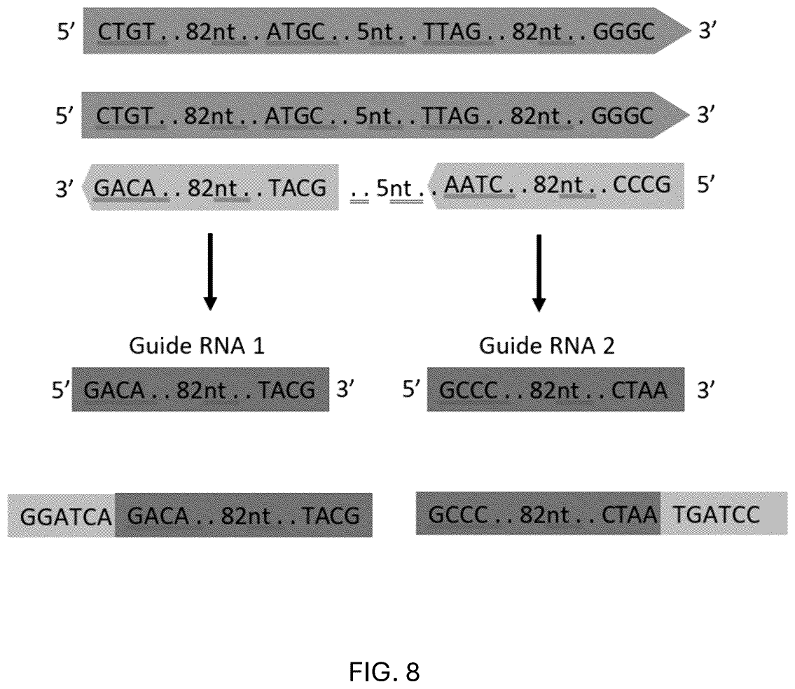

Guide RNA sequence design for use in the split ribozyme biosensor system disclosed herein is illustrated in FIG. 8. Examples of gRNA sequences used can be found in SEQ ID NOs: 15-25. For example of the use of gRNA with the present invention, gRNA 05 (SEQ ID NO: 19) and gRNA 06 (SEQ ID NO: 20) are used in construct pXYB147 of FIG. 3a targeting AT3G20840. In the detection of RNA using the split ribozyme biosensor of the present disclosure where sfGFP is used as a reporter, a fluorescence microscope is necessary, which does not allow detection in live tissues or in the field. In order to detect RNA of target genes in live tissue, sfGFP with a GFP-like protein (eYGFPuv), which was recently utilized for in vivo visualization of transgene expression through the use of a UV flashlight. Ribozyme splicing efficiency was lower than desired (see Example 4 below). Attachment of the eYGFPuv through a self-cleavage peptide eliminated the need to re-engineer specific split sites based on the reporter protein. Therefore, combining a self-cleavage peptide in the ribozyme-mediated transcript splicing system created a modular platform to convert RNA signals to different protein outputs. The disclosed “ribozyme_sfGFP-2A-protein” design produces functional proteins, this configuration providing potential uses of designing new split ribozyme-based systems with any functional protein as an output for RNA-based genome engineering at cellular level in plants.

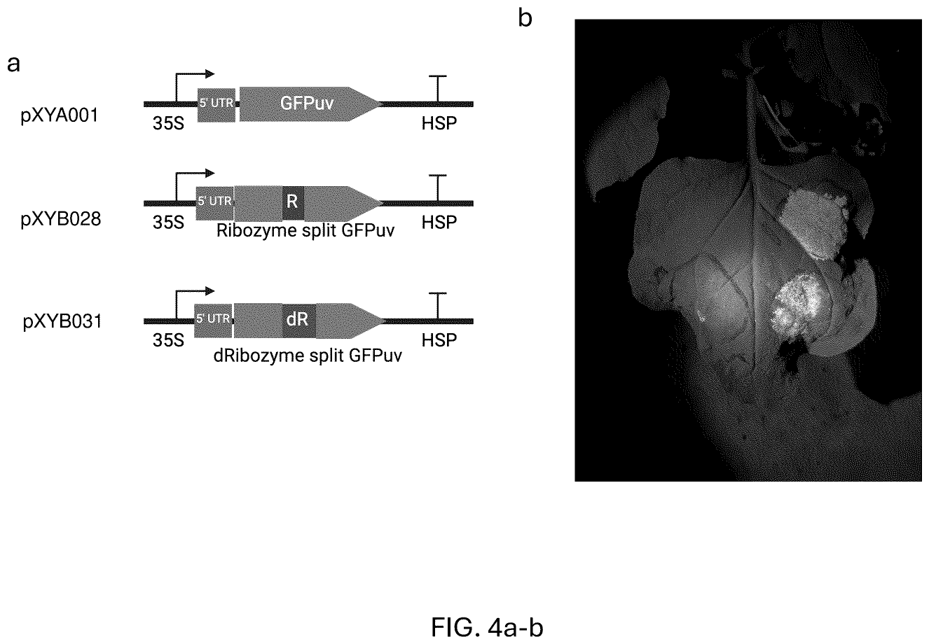

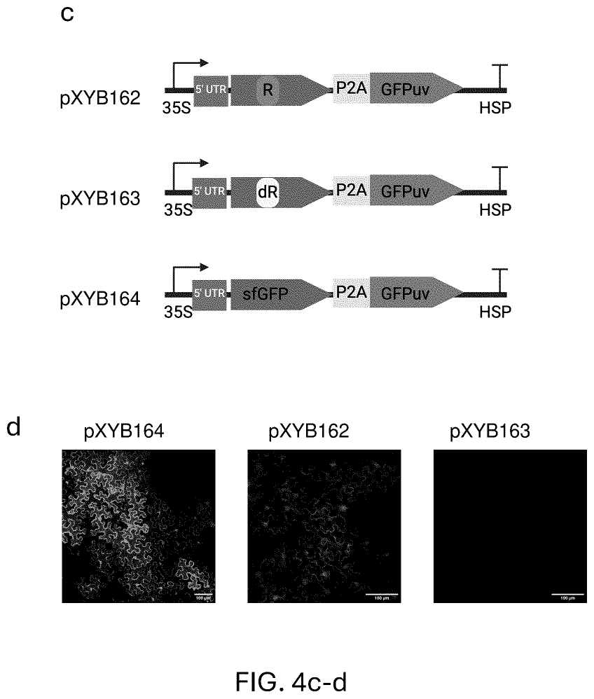

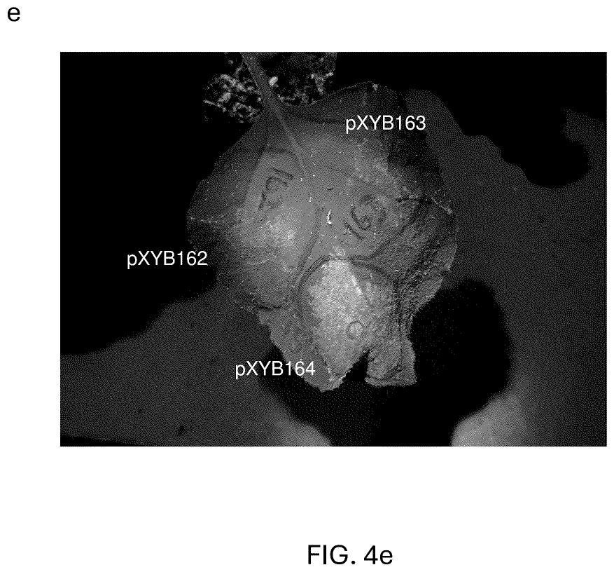

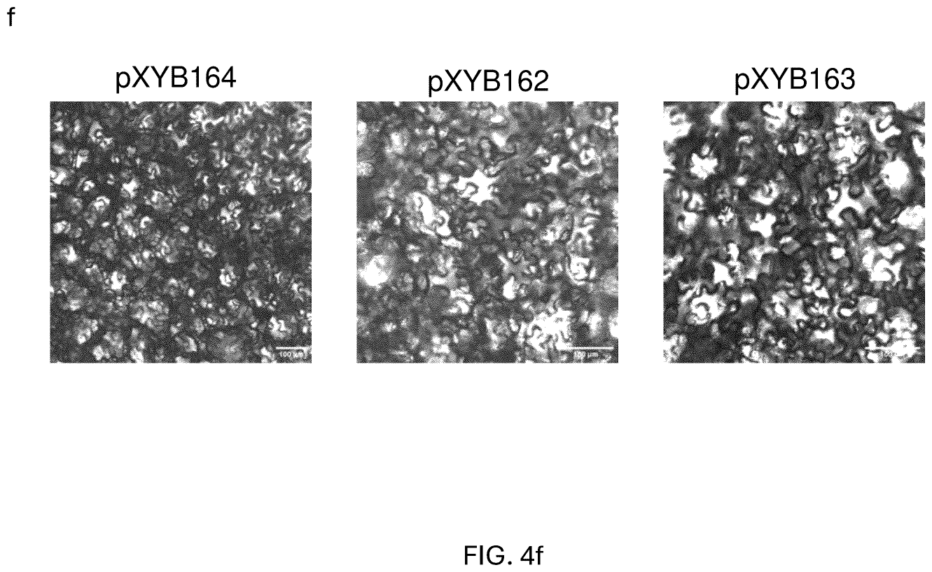

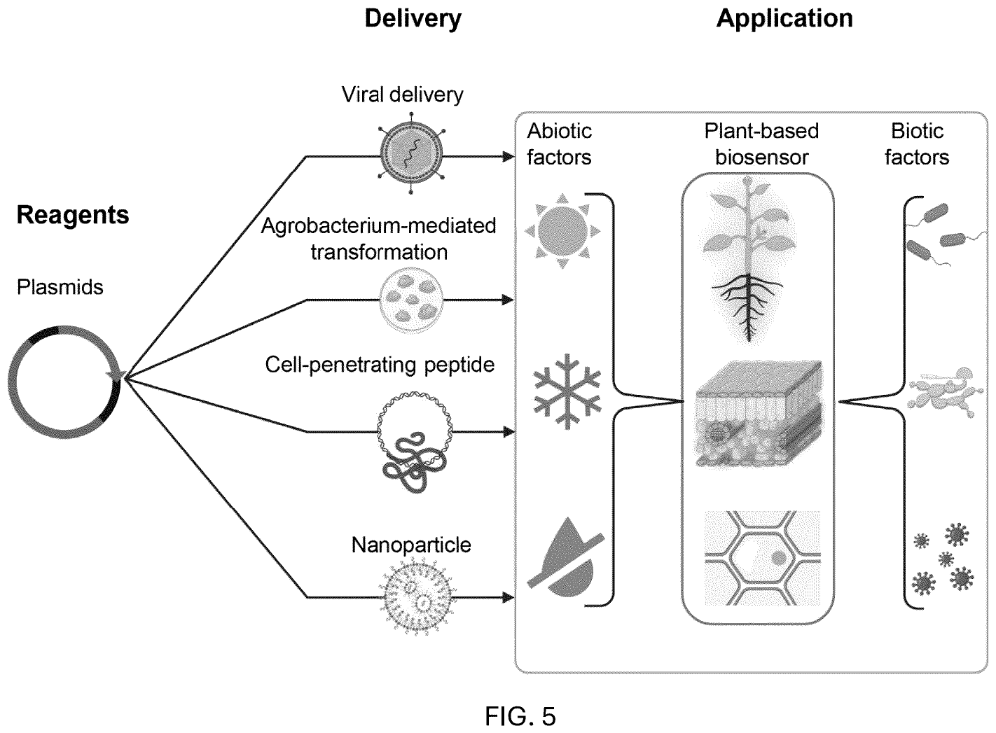

In some embodiments, the second polynucleotide further comprises a nucleotide sequence encoding a self-cleaving peptide linked to a second protein, wherein upon translation, the first protein and the second protein are separated. Self-cleaving peptides are short viral peptides that can mediate cleavage between two connected peptides during translation, effectively separating them into two distinct protein products. Common examples of self-cleaving peptides include P2A (porcine teschovirus-1), E2A (equine rhinitis A virus), F2A (foot-and-mouth disease virus), and T2A (thosea asigna virus). In some embodiments, the self-cleaving peptide is P2A and comprises the amino acid sequence of ATNFSLLKQAGDVEENPGP (SEQ ID NO: 28). In some embodiments, the self-cleaving peptide is E2A and comprises the amino acid sequence of QCTNYALLKLAGDVESNPGP (SEQ ID NO: 29). In some embodiments, the self-cleaving peptide is F2A and comprises the amino acid sequence of VKQTLNFDLLKLAGDVESNPGP (SEQ ID NO: 30). In some embodiments, the self-cleaving peptide is T2A and comprises the amino acid sequence of EGRGSLLTCGDVEENPGP (SEQ ID NO: 31). In some embodiments, the linker GSG is added on to the N-terminal of a 2A peptide to assist in efficiency. In some embodiments, the self-cleaving peptide P2A is encoded by a nucleic acid comprising the sequence of SEQ ID NO: 8. An exemplary embodiment of a split ribozyme biosensor system comprising a self-cleaving peptide is shown as pXYB162 in FIG. 4c. Where, from 5′ to 3′, the construct comprises a 35S promoter, 5′UTR, a first fragment of a sfGFP reporter, ribozyme, second fragment of sfGFP reporter, self-cleavage peptide, GFPuv, and HSP terminator.

In some embodiments, the polynucleotides further comprise linkers between the specific nucleotide sequences. In some embodiments, the first polynucleotide comprises a linker nucleotide sequence between the promoter and the 5′ UTR of the first expression cassette. In some embodiments, the first polynucleotide comprises a linker nucleotide sequence between the first P1 loop sequence and the first guide RNA sequence. In some embodiments, the second polynucleotide comprises a linker nucleotide sequence between the second gRNA and the second P1 loop sequence.

In some embodiments, the second protein is a reporter protein. In some embodiments, the first protein is a reporter protein and the second protein is a reporter protein, and the first and second reporter proteins are different reporter proteins.

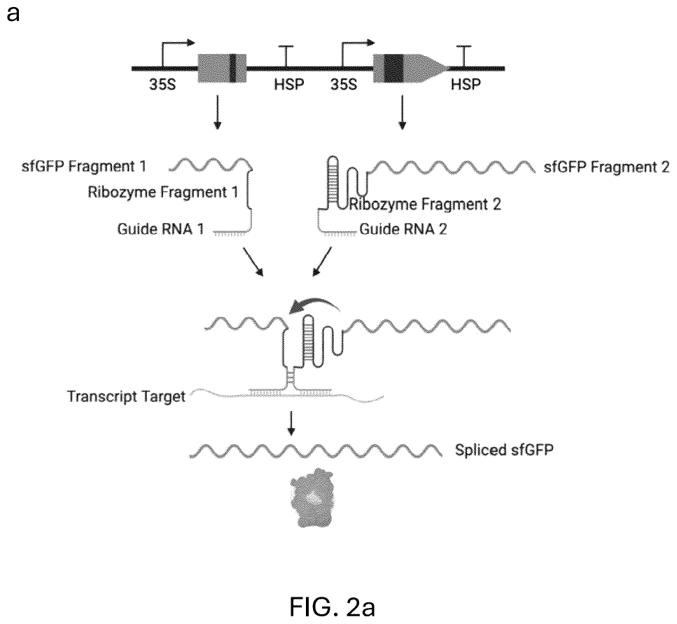

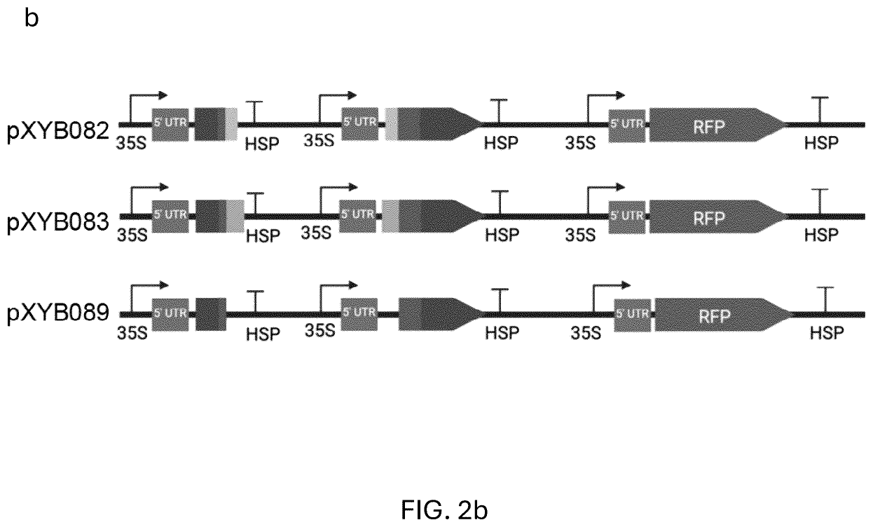

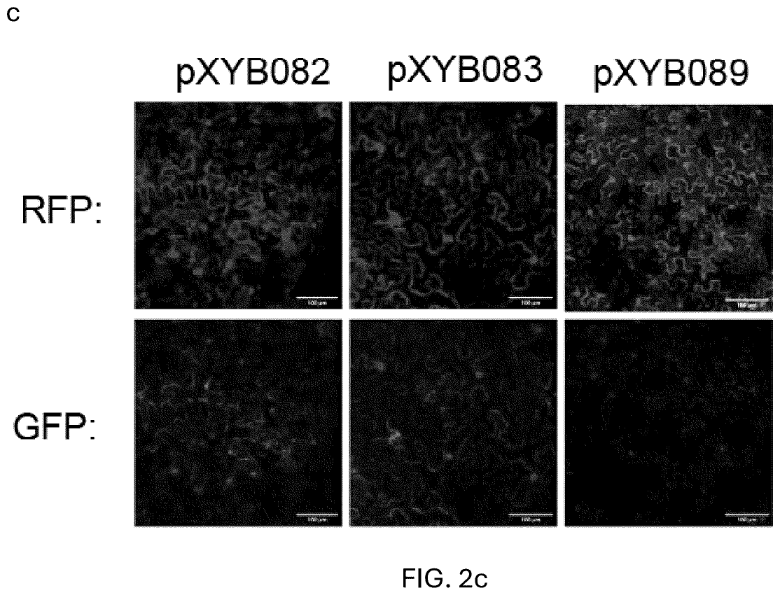

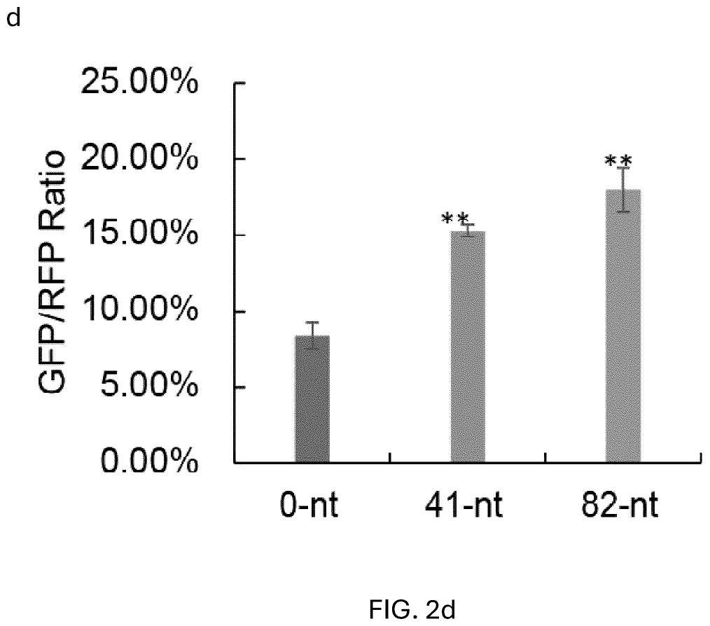

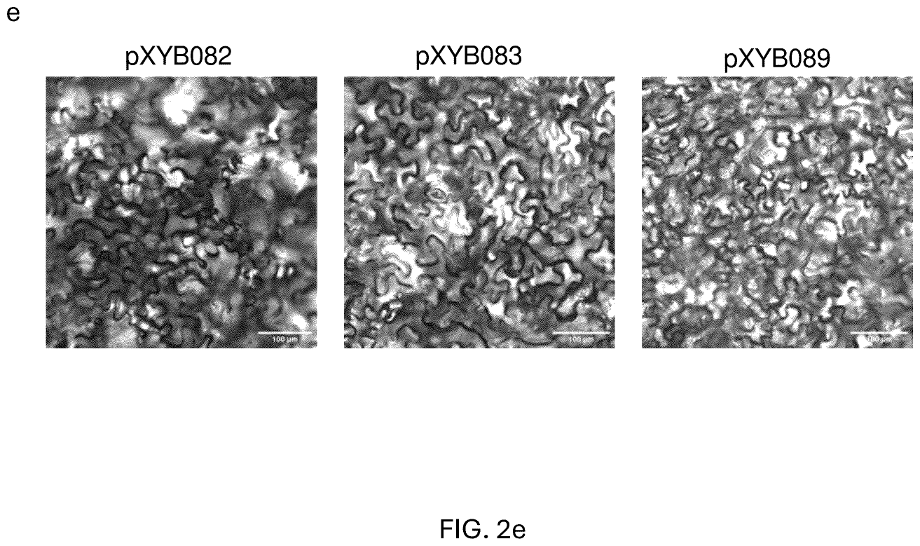

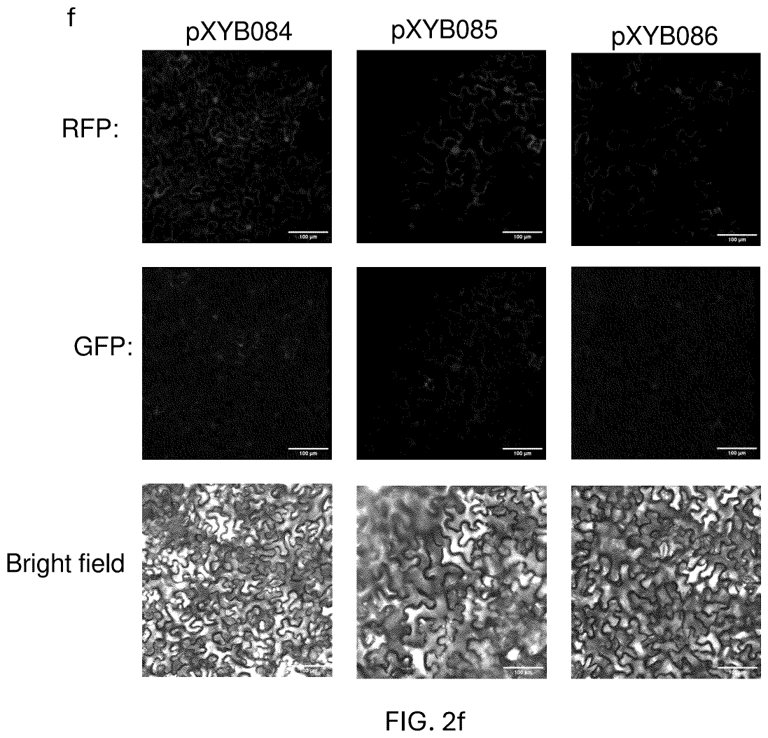

In some embodiments, the biosensor system further comprises a third polynucleotide sequence comprising a promoter sequence, a 5′ untranslated sequence, a nucleotide sequence encoding a third protein, and a terminator sequence. In some embodiments, the third protein is a reporter protein. An example of such an embodiment including the third protein that is a reporter protein is shown in FIG. 2b. Such an embodiment provides independent indication of successful transfection of the expression cassette.

In some embodiments, where the system comprises multiple reporter proteins, the reporter proteins are different reporter proteins.

In some embodiments, the reporter protein is a fluorescent protein reporter or bioluminescence reporter protein (luciferase).

In some embodiments, the two expression cassettes are placed on the same vector, i.e. cassette 1 and cassette 2 are on the same vector. In some embodiments, the two expression cassettes on the same vector are separated by a linker nucleotide sequence between the terminator sequence of the first expression cassette and the promoter sequence of the second expression cassette.

An exemplary embodiment of a DNA construct of the split ribozyme biosensor system disclosed herein is pXYB147. pXYB147 is a construct comprising 82 bp gRNAs that target the Arabidopsis thaliana gene, AT3G20840. The construct design of pXYB147 is shown in FIG. 2b. The pXYB147 construct is laid out in the order: 35S::5′UTR::sfGFPn-P1_loop_01-stem01-gRNA05::HSP---35S::gRNA06-stem_02-P1_loop_02-IGS-Ribozyme-sfGFPn_Y66_02::HSP. It will be understood by one of skill in the art that the target RNA, RNA of interest, target gene, or gene of interest of the disclosed split ribozyme biosensor system can be any target RNA.

### Methods of Examining In Vivo RNA Expression in Plants

Another aspect of the current disclosure is related to methods for examining in vivo RNA expression in plants, plant cells, or plant tissues, the method comprising introducing the biosensor system described herein into a plant, plant cell, or plant tissue to create a genetically modified plant, plant cell, or plant tissue.

In some embodiments, introducing the split ribozyme biosensor system into a plant, plant cell, or plant tissue comprises delivering the expression cassettes of the system into the plants, plant cells, or plant tissues. Methods of transforming plants are known in the art. In some embodiments, the transformation is a stable transformation. As used herein, stable transformation means that the gene will be fully integrated into the host genome and is expressed continuously. The gene in a stable transformation will also be expressed in later generations of the plant. There are numerous proven genetic transformation methods in the art that can stably introduce new genes into the nuclear genomes of different plant species. Exogenous genes can be delivered to plant cells by Agrobacterium, particle bombardment/gene gun, electroporation, the pollen tube pathway, and other known mediated delivery methods.

One method of plant transformation known in the art is Agrobacterium-mediated plant transformation. As a genus, Agrobacterium can transfer DNA to a remarkably broad group of organisms including numerous dicot and monocot angiosperm species and gymnosperms. Additionally, Agrobacterium can transform fungi, yeasts, ascomycetes, and basidiomycetes. This is known as the most common method of plant transformation. The Agrobacterium's DNA is engineered to carry the desired gene, and the Agrobacterium naturally transfers its T-DNA into the plant cells.

A subtype of Agrobacterium transformation is the floral dip method. In this method, transformation of female gametes is accomplished by simply dipping developing Arabidopsis inflorescences for a few seconds into a 5% sucrose solution containing 0.01-0.05% (vol/vol) Silwet L-77 and resuspended Agrobacterium cells carrying the genes to be transferred. Treated plants are allowed to set seed which are then plated on a selective medium to screen for transformants. A transformation frequency of at least 1% can be routinely obtained and a minimum of several hundred independent transgenic lines generated from just two pots of infiltrated plants (20-30 plants per pot) within 2-3 months.

Another method of plant transformation is particle bombardment (also known as gene gun). This method involves coating the target gene on the surface of gold or tungsten powder to construct a DNA-coated microcarrier. High-pressure helium pulses accelerate the DNA-coated microcarrier into the gas acceleration tube using an electric discharge or a pressurized helium gas stream. These particles gain sufficient momentum to pierce recipient cells at high speed, while the target gene coated on the outside remains in the cell and is eventually integrated into the plant's chromosome, producing the transformed plant.

Another method of transformation of plant cells is electroporation. Electroporation uses short, high-field electrical pulses to create transient pores in the plasma membrane of target cells, increasing the permeability of the host cell membrane. Under an optimal electrical pulse, these pores can be resealed, restoring the cells to their original state. Compared to Agrobacterium and particle-bombardment-mediated plant transformation, electroporation-mediated transformation has the advantages of rapid application, low cost, and a highly stable transformation rate.

Another known method of transforming plant cells is the pollen tube pathway-mediated transformation. The pollination process of higher plants, pollen forms the pollen tube after germination on the stigma surface and extends to the ovule along the style, and the pollen nucleus passes through the pollen tube to fertilize the ovule. Pollen-tube-mediated plant genetic transformation entails removing the stigma from the recipient plant immediately after pollination and adding exogenous DNA solution dropwise to the recipient plant's severed style. The exogenous DNA is transported to the recipient plant's ovary by pollen tube growth, where it is integrated with the undivided but fertilized recipient egg, resulting in the exogenous DNA being integrated into the recipient's genome at the embryogenic stage and being present in the transformed seed.

Liposome-Mediated Plant Genetic Transformation is also known in the art. Liposomes are spherical vesicles composed of one or more phospholipid bilayer membranes, ranging in size from 30 nm to several μm, and composed of cholesterol and natural nontoxic phospholipids. According to the size and number of bilayer membranes, liposomes can be divided into two types: multilamellar vesicles (MLV) and unilamellar vesicles. The latter is further classified into large unilamellar vesicles (LUV) and small unilamellar vesicles (SUV). Liposome-mediated transformation can introduce exogenous DNA into protoplasts through plasma membrane fusion or protoplast endocytosis. Liposomes and DNA are mixed and incubated to form a DNA-lipid complex, which is subsequently mixed with protoplast suspension (supplemented with PEG), and the desired DNA is introduced into the target protoplast through liposome-protoplast fusion or endocytosis. The positively charged liposome is attracted to the negatively charged DNA and the cell membrane, enabling adhesion of the liposome to the protoplast surface, followed by the incorporation of the liposome and protoplast at their binding sites, and finally releasing the plasmid into the target cells.

Silicon-Carbide-Whisker-Mediated Transformation is also known as a method of transforming plant cells. Silicon carbide whiskers (SCWs) consist of needle-like microwhiskers with a diameter of about 0.5 μm and a length of about 10-80 μm. The whiskers are tough and easily cleaved, resulting in sharp cutting edges that pierce the cell wall and eventually the cell nucleus. SCW-mediated plant genetic transformation is achieved by placing suspended cells or embryogenic calli and DNA in a centrifuge tube containing SCW, which cannot bind to DNA due to its negatively charged surface. Through vortexing, SCWs can create needle-like pores on the cell membrane through which exogenous DNA can enter the target cells

In microinjection-mediated plant genetic transformation, DNA is injected into a single plant nucleus or cytoplasm using a glass microcapillary injection pipette. In this technique, the target cell is fixed under a microscope; there are two micromanipulators, one of which is the holding pipette that fixes the cell and the other is a microcapillary tube containing a small amount of DNA solution to penetrate the cell membrane or nuclear membrane. Through injection, the DNA is transferred into the cytoplasm/nucleus of plant cells or protoplasts using the microcapillary pipette (0.5-10 μm at the tip), and the transformed cells are cultured and grown into transgenic plants after gene transfer is completed.

In some embodiments, the first protein of the biosensor system is a reporter protein, the method further comprising detecting the reporter protein as indicative of the expression of the target RNA. In some embodiments, the second protein of the biosensor system is a reporter protein, the method further comprising detecting the reporter protein as indicative of the expression of the target RNA. In some embodiments, the detecting the reporter protein occurs through optical imaging or electrochemical imaging. In some embodiments, the optical imaging is selected from fluorescence imaging, bioluminescence imaging, RGB imaging, hyperspectral imaging, and Raman imaging.

In some embodiments, the method further comprises subjecting the genetically modified plant, plant cells, or plant tissue to experimental conditions before the step of detecting the reporter protein. The experimental conditions comprise abiotic stress, biological stress, and improved growth conditions. Abiotic stresses are known in the art and examples of commonly used abiotic stresses include temperature, drought, salinity, or heavy metal stresses. Biotic stresses are known in the art and examples of commonly used biotic stresses include stresses caused by fungi, bacteria, viruses, insects, or herbivores. Improved growth conditions are known in the art and examples of improved growth conditions include optimal light, temperature, moisture, nutrients, and airflow.

### Genetically Modified Plants, Plant Tissues, and Plant Cells

Another aspect of the current disclosure is directed to a genetically modified plant, plant cell, or plant tissue comprising the biosensor system described herein.

In some embodiments, the plant is an herbaceous plant. In some embodiments, the herbaceous plant is selected from the group comprising Arabidopsis thaliana, Avena sativa, Brachypodium distachyon, Brassica spp., Cannabis sativa, Glycine max, Gossypium spp., Helianthus annuus, Hordeum vulgare, Medicago sativa, Miscanthus giganteus, Nicotiana spp., Oryza sativa, Secale cereale, Solanum spp., Panicum virgatum, Saccharum officinarum, Setaria italica, Sorghum bicolor, Triticum aestivum, and Zea mays.

In some embodiments, the plant is a woody plant. In some embodiments, the woody plant is selected from the group comprising Citrus spp., Eucalyptus spp., Malus spp., Populus spp., Prunus spp., Pyrus spp., Salix spp., and Vitis vinifera.

### Split Ribozyme Biosensor Kits

Another aspect of the current disclosure is directed to a kit comprising: a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein. In some embodiments, the kit further comprises a third nucleotide sequence encoding a second protein. In some embodiments, the polynucleotide sequences of the kit can be delivered into plants or plant cells using various methods, including biological methods mediated by bacteria (Agrobacterium/Rhizobium) or viral vectors, physical methods mediated by gene gun or electroporation, and chemical methods mediated polyethylene glycol (PEG), polymers, lipids, or cell-penetrating peptides.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example 1: Evaluating Ribozyme Splicing Activity in Plants

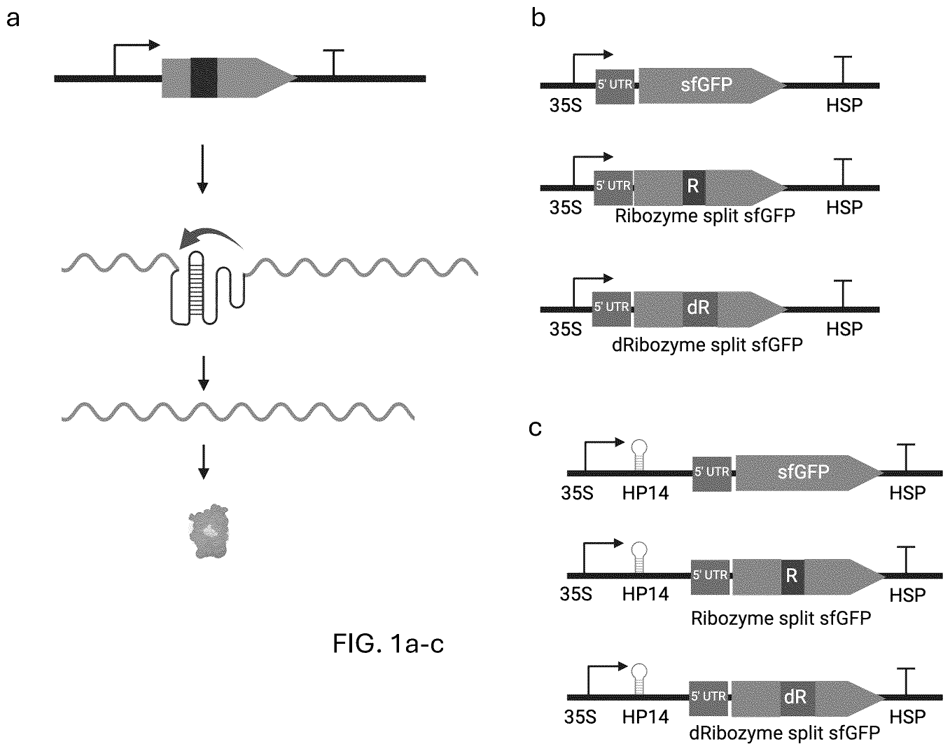

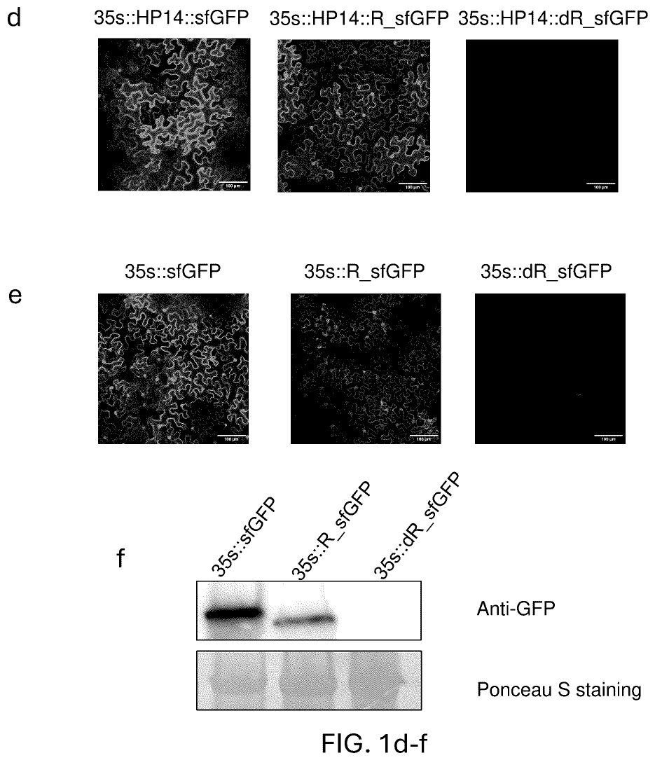

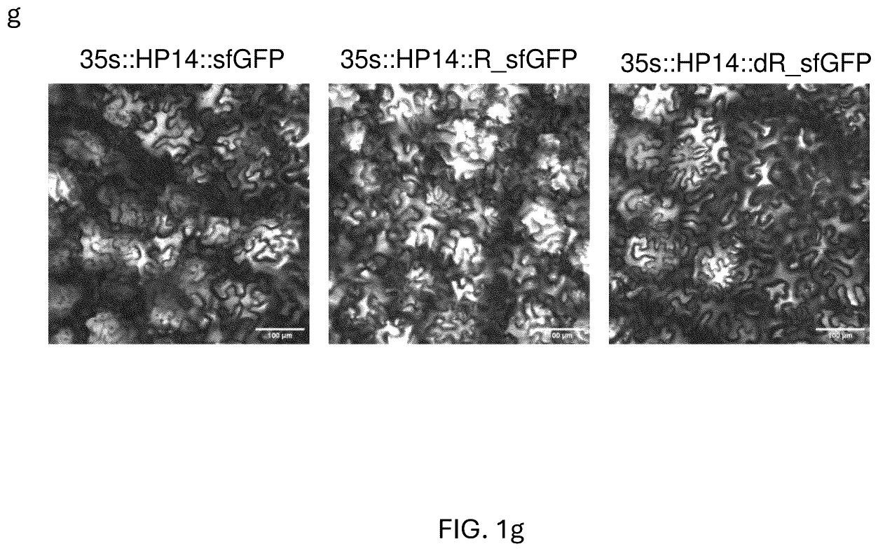

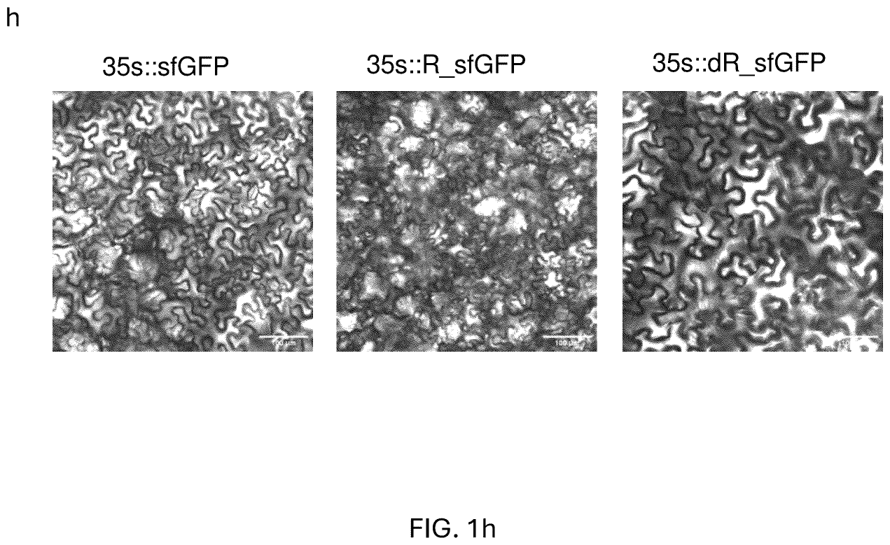

To evaluate ribozyme splicing activity in plants, a fluorescence-based splicing assay originally developed in Escherichia coli was used (Gambill et al., 2023). Specifically, the self-splicing Tetrahymena thermophila ribozyme was inserted into the coding sequence (CDS) of sfGFP, interrupting the translation of the functional protein (Gambill et al., 2023) (FIG. 1a). A functional sfGFP transcript can only be translated when the ribozyme excises itself from the surrounding exons and joins the split segments together (Gambill et al., 2023) (FIG. 1a). The 35S promoter was used to drive expression of sfGFP inserted with T. thermophila ribozyme with and without an RNA stable structure hairpin 14 (HP14) (Carrier and Keasling, 1999) (FIG. 1b, c). These results demonstrated that the ribozyme splicing activity functions efficiently for splicing of the sfGFP transcript in N. benthamiana, as visualized under a fluorescence microscope/stereoscope (FIGS. 1d, e, g, and h). Removing the HP14 hairpin in the original design used in E. coli (Gambill et al., 2023) produced weaker sfGFP fluorescence output of the system in N. benthamiana compared with the design containing the HP14 (FIG. 1d vs 1e), indicating that this element can enhance the ribozyme splicing activity in plants. Western blot analysis confirmed that the sfGFP transcript fragments flanking the ribozyme were able to reassemble through ribozyme-mediated splicing and be translated into full-length sfGFP (FIG. 1f). The intensity of anti-GFP band from the reassembled sfGFP (35S::R_sfGFP) is weaker compared with the positive control (35S::sfGFP) (FIG. 1f), indicating that the amount of reconstituted sfGFP protein from ribozyme-mediated splicing is less than that of the positive control (35S::sfGFP).

### Example 2: Engineering a Split Ribozyme System that Converts RNA Signals to a Visible Reporter

Next, we aimed to convert RNA signals into orthogonal protein outputs in plants. The core concept of the split ribozyme system is to attach gRNA sequences to each ribozyme fragment for complementation. These gRNA sequences were engineered to base pair with a transcript and thereby, in the presence of the RNA target, the ribozyme fragments are brought together, enabling splicing to occur (FIG. 2a). The split site was tested at nucleotide 15 validated in E. coli (Gambill et al., 2023) in N. benthamiana using Agrobacterium-mediated leaf infiltration. Specifically, two plasmid vectors were constructed (pXYB082, pXYB083), each carrying three expression cassettes. One cassette encodes the sequence of a red fluorescence protein (RFP), while the other two cassettes contain a fragment of sfGFP fused to a fragment of the Tetrahymena ribozyme linked to a gRNA targeting the RFP CDS (FIG. 2b). The two plasmid vectors are identical with the exception of gRNAs of differing lengths, 41-nt and 82-nt in pXYB082 and pXYB083, respectively. After leaf infiltration, green sfGFP fluorescence signal was detected in the N. benthamiana leaf tissue infiltrated with pXYB082 (containing a 41-nt gRNA) and pXYB083 (containing an 82-nt gRNA) (FIGS. 2c and 2e). Because low “off” state fluorescence from the split ribozyme system was consistently detected in E. coli, we then evaluated the potential background noise of split ribozyme system in plant using the plasmid vector (pXYB089) without any gRNAs. Weak sfGFP signal was also detected in the leaf tissue infiltrated with the negative control plasmid vector (pXYB089), but the GFP signal is significantly lower compared with split-ribozyme systems with gRNAs (FIGS. 2c and 2d), indicating that the split ribozyme-based biosensor can be used to detect RNAs in plant cells.

To investigate if the increased gRNA length could enhance the florescent signal from the split-ribozyme system, three more gRNAs with lengths of 123-nt (in pXYB084), 164-nt (in pXYB085) and 325-nt (in pXYB086) were tested. Surprisingly, unlike the results seen in E. coli (Gambill et al., 2023), there was no increase in the sfGFP fluorescence signal produced from split ribozyme system with increased gRNA length (FIG. 2f).

### Example 3. Using the Split Ribozyme-Based Biosensor System to Detect Different RNA Signals in Plants

The split ribozyme-based biosensor system was tested through targeting an Arabidopsis thaliana gene, PLT1 (AT3G20840), which encodes an AP2 transcription factor expressed in the root quiescent center (Nawy et al., 2005) using N. benthamiana leaf infiltration. The AT3G20840 CDS was cloned under a strong promoter 35S (pXYB151). A biosensor vector was created with 82-nt gRNAs targeting the AT3G20840 CDS (pXYB147) (FIG. 3a). Green sfGFP signal was detected in the N. benthamiana leaf tissue co-infiltrated with pXYB147 and pXYB151, whereas sfGFP signal was not detected in the leaf tissue infiltrated with pXYB147 or pXYB151 alone (FIGS. 3b and 3e). Furthermore, the split ribozyme-based biosensor was used to detect infection by tobacco rattle virus (TRV). Two biosensor vectors (pXYB155 and pXYB156) were engineered with 82-nt gRNAs targeting different regions of the TRV RNA sequence (FIG. 3a). sfGFP signals were detected in the N. benthamiana leaf tissues co-infiltrated with TRV and one of the two biosensor constructs (pXYB155 and pXYB156). No sfGFP signal was detected in the leaf tissues infiltrated with TRV alone or the biosensor (pXYB155 or pXYB156) alone (FIGS. 3c and 3f). These results indicate that the split ribozyme-based biosensor system can be used in vivo to detect RNAs derived from diverse sources using transient expression approaches.

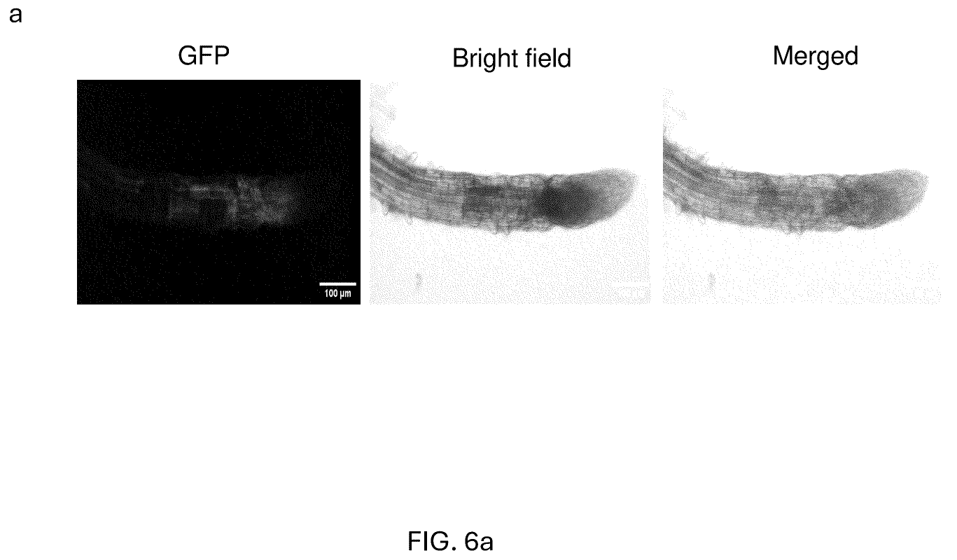

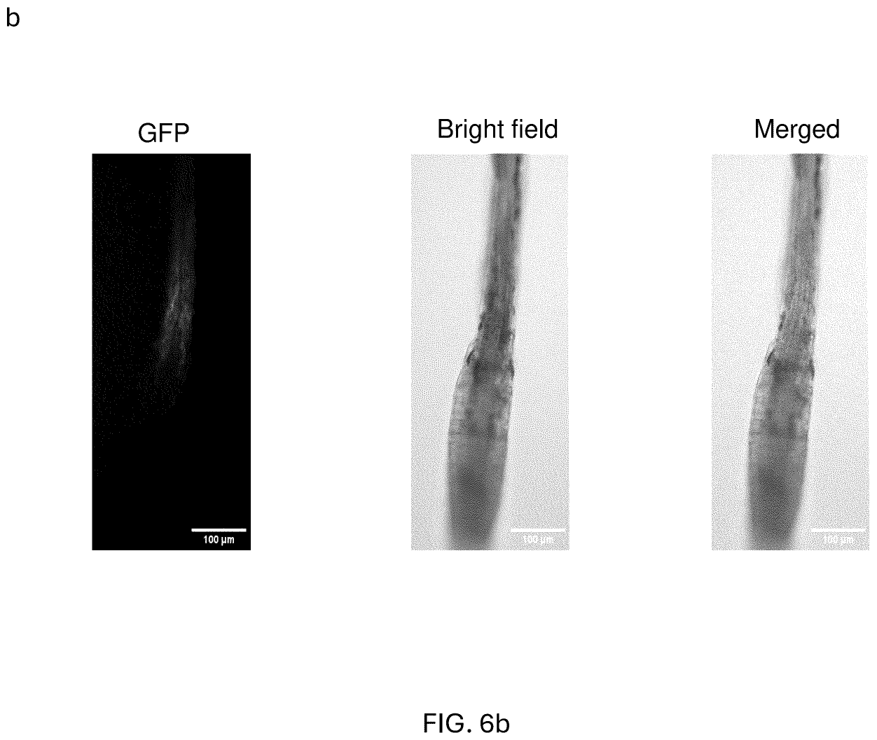

Based on the success of utilizing the split ribozyme-based biosensor system to detect RNAs through transient expression in N. benthamiana leaves, the biosensor construct pXYB147 (FIG. 3a) was engineered with 82-nt gRNAs targeting the AT3G20840 CDS into A. thaliana using Agrobacterium-mediated stable transformation. The sfGFP signal was detected in the roots of three T1 transgenic Arabidopsis plants (FIG. 3d, FIGS. 6a and 6b), indicating that the split ribozyme-based biosensor system can also be used to detect endogenous gene expression at the cellular level in plants with high specificity.

### Example 4. Engineering a Split Ribozyme-Based Platform for Orthogonal Protein Outputs

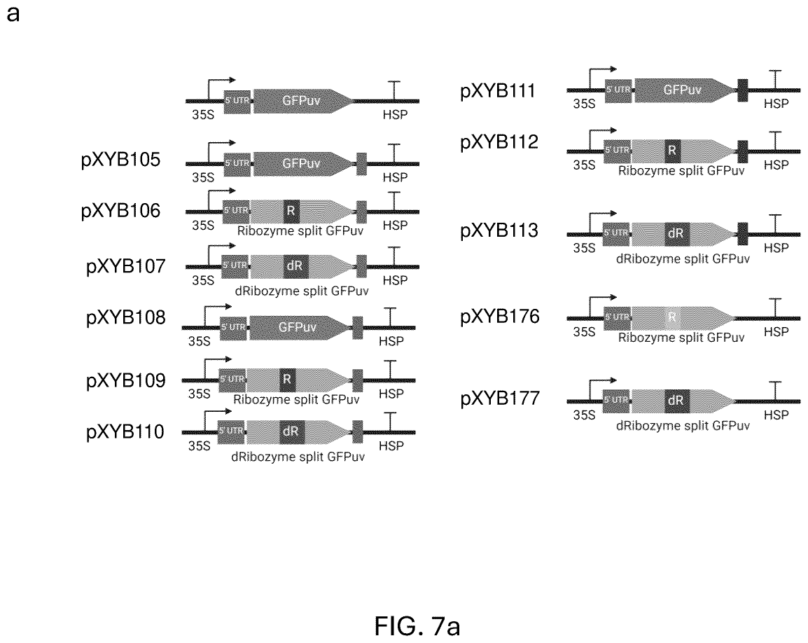

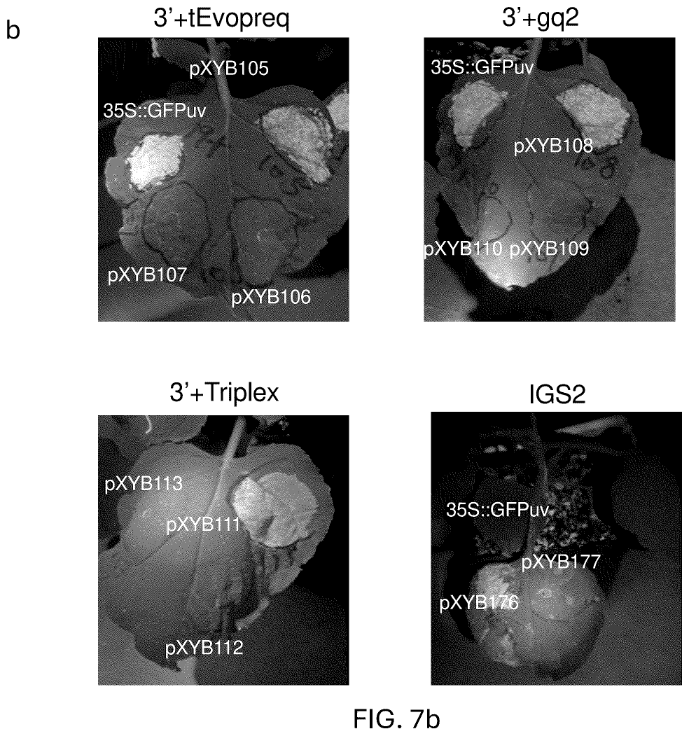

RNA detection using the split ribozyme-based biosensor system with sfGFP as a reporter requires the use of a fluorescence microscope, which is technically demanding and time consuming and does not allow detection in live tissues. To address this limitation, sfGFP was replaced with a GFP-like protein (eYGFPuv), which was recently utilized for in vivo visualization of transgene expression under a UV flashlight (Yuan et al., 2021). First, we tested if ribozyme-mediated splicing can reassemble two partial fragments of the eYGFPuv transcripts in plant cells using N. benthamiana leaf infiltration. Ribozyme-mediated cleavage at the 5′ splice site depends on formation of a base-paired helix P1 between the Internal Guide Sequence (IGS) and sequences adjacent the splice site, and the presence of a U.G “wobble” base-pair positioned 4-6 residues from the base of this helix defines the susceptible phosphodiester bond (Köhler et al., 1999). Therefore, the ribozyme is inserted immediately downstream of an uracil where the IGS should start with a guanine and the remaining IGS sequence is in reverse complementation to the first 5 base pairs of the P1 helix (Köhler et al., 1999). Following these principles, different insertion sites of eYGFPuv CDS were tested and alternate IGS for different target sites designed. The eYGFPuv with the ribozyme inserted after the last amino acid of Y72 showed a strong fluorescence signal under a UV flashlight (FIGS. 4a and 4b). However, only one of six replicates showed desirable eYGFPuv signal, indicating correct splicing efficiency was low. To enhance the ribozyme splicing efficiency, the IGS of the original ribozyme design was modified, known to pair with the P1 helix and tested different RNA stable motif in eukaryotes (FIG. 7). However, none of those optimizations efficiently enhanced the ribozyme splicing activity in eYGFPuv (FIG. 7). These results suggest that ribozyme splicing efficiency is different under alternate transcript contexts in plants. Variable ribozyme-mediated splicing efficiency was also observed in mammalian cells (Hasegawa et al., 2004; Rogers et al., 2002). Hence, a modular platform allowing for simple exchange of the output protein would be helpful for avoiding the inconsistent ribozyme splicing activity in different contexts.

To bypass the need to redesign ribozyme insertion sites for different protein outputs (e.g., eYGFPuv), a construct (pXYB162) was created with eYGFPuv attached via the 2A self-cleaving peptide P2A to sfGFP (FIG. 4c), which was optimized for ribozyme-mediated splicing. Because the ribozyme contains stop codons in-frame with sfGFP and eYGFPuv, the production of these two functional proteins requires self-splicing of the ribozyme. The sfGFP signal was detected in the N. benthamiana leaf tissue infiltrated with the construct pXYB162 (containing two sfGFP fragments flanking the ribozyme) although the signal was weaker than that in leaf tissue infiltrated with the positive control construct pXYB164 (containing intact sfGFP); no sfGFP signal was detected in the leaf tissue infiltrated with the negative control construct pXYB163 (containing two sfGFP fragments flanking a catalytically dead G264A mutant ribozyme with IGS and P1 loop removed) (FIGS. 4d and 4f). This result indicates that the eYGFPuv attachment did not affect the reassembly of sfGFP fragments through ribozyme-mediated transcript splicing.

While the eYGFPuv fluorescence signal detected in N. benthamiana leaf tissue infiltrated with pXYB162 was much weaker than that in leaf tissue infiltrated with the positive control construct pXYB164, the results demonstrate that ribozyme-mediate splicing and 2A self-cleaving peptide can be combined and still produce functional proteins (FIG. 4e).

### Example 5: General Materials and Methods

### Plant Materials

Arabidopsis wild-type Col-0 plants and Nicotiana benthamiana (GenBank: PRJNA170566) plants were grown in the soil in the growth chamber at 21° C. under 100 μmol m-2 s-1 white light with 12-h light/12-h dark photoperiod.

### Plasmids Construction

The DNA constructs used in this study are listed in Table 1. The constructs were created via Gibson assembly using NEBuilder® HiFi DNA Assembly master mix (NEB, Cat. No. E2621) or Golden Gate assembly using the Modular Vector Library cloning Kit from Dr. Dan Voytas' lab at the University of Minnesota (Čermák et al., 2017). Sequence information for the DNA constructs is provided in Tables 2-5. The gRNA sequences design is illustrated in FIG. 8. Sequences were synthesized by TWIST Bioscience (South San Francisco, United States).

TABLE 1

| Plasmid | FIGS. | Description | Plasmid design |
|---|---|---|---|
| pXYB033 | FIG. 1 | sfGFP expression | 35S::5′UTR::sfGFP::HSP |
| pXYB034 | FIG. 1 | Ribozyme split sfGFP | 35S::5′UTR::sfGFPn_Y66-Ribozyme- |
| | | expression | 2nd_sfGFPc::HSP |
| pXYB035 | FIG. 1 | Deactivated ribozyme split | 35S::5′UTR::sfGFPn_Y66-dRibozyme- |
| | | sfGFP expression | 2nd_sfGFPc::HSP |
| pAXY0001 | FIG. 4 | GFPuv expression | 35S::5′UTR::GFPuv::HSP |
| pXYB028 | FIG. 4 | Ribozyme split GFPuv | 35S::5′UTR::GFPuvn_Y72-Ribozyme- |
| | | expression | 2nd_GFPuvc::HSP |
| pXYB031 | FIG. 4 | Deactivated ribozyme split | 35S::5′UTR::GFPuvn_Y72-dRibozyme- |
| | | GFPuv expression | 2nd_GFPuvc::HSP |
| pXYB082 | FIG. 2 | Plant RNA Vision constructs | 35S::5′UTR::sfGFPn_Y66-P1_loop_01- |
| | | with 41 bp gRNAs together | stem01-gRNA01::HSP - - - |
| | | with RFP input | 35S::5′UTR::gRNA02-stem_02- |
| | | | P1_loop_03-internal guide sequence- |
| | | | Ribozyme-sfGFPn_Y66::HSP - - - |
| | | | 35S::RFP::HSP |
| pXYB083 | FIG. 2 | Plant RNA Vision | 35S::5′UTR::sfGFPn_Y66-P1_loop_01- |
| | | constructs with 82 bp | stem01-gRNA03::HSP - - - |
| | | gRNAs together with RFP | 35S::5′UTR::gRNA04-stem_02- |
| | | input | P1_loop_03-internal guide sequence- |
| | | | Ribozyme-sfGFPn_Y66::HSP - - - |
| | | | 35S::RFP::HSP |
| pXYB089 | FIG. 2 | Plant RNA Vision constructs | 35S:5′UTR::sfGFPn_Y66-P1_loop_01- |
| | | with no gRNAs together with | stem01::HSP - - - 35S::5′UTR::stem_02- |
| | | RFP input | P1_loop_03-internal guide sequence- |
| | | | Ribozyme-sfGFPn_Y66::HSP - - - |
| | | | 35S::RFP::HSP |
| pXYB147 | FIG. 3 | Plant RNA Vision constructs | 35S::5′UTR::sfGFPn_Y66-P1_loop_01- |
| | | with 82 bp gRNAs targeting | stem01-gRNA05::HSP - 35S::gRNA06- |
| | | AT3G20840 | stem_02-P1_loop_03-internal guide |
| | | | sequence- Ribozyme-sfGFPn_Y66::HSP |
| pXYB151 | FIG. 3 | Constitutive RNA input | 35S::AT3G20840::NOS |
| pXYB155 | FIG. 3 | Plant RNA Vision constructs | 35S:5′UTR::sfGFPn_Y66-P1_loop_01- |
| | | with with 82 bp gRNAs | stem01-gRNA13::HSP - - - |
| | | targeting TRV | 35S::gRNA14-stem_02-P1_loop_03- |
| | | | internal guide sequence- Ribozyme- |
| | | | sfGFPn_Y66::HSP |
| pXYB156 | FIG. 3 | Plant RNA Vision constructs | 35S:5′UTR::sfGFPn_Y66-P1_loop_01- |
| | | with with 82 bp gRNAs | stem01-gRNA15::HSP - - - |
| | | targeting TRV | 35S::gRNA16-stem_02-P1_loop_03- |
| | | | internal guide sequence- Ribozyme- |
| | | | sfGFPn_Y66::HSP |
| pXYB162 | FIG. 4 | Ribozyme split sfGFP linked | 35S::5′UTR::sfGFPn_Y66-Ribozyme- |
| | | with GFPuv via T2A | 2nd_sfGFPc-T2A- GFPuv::HSP |
| | | expression | |
| pXYB163 | FIG. 4 | Deactivated ribozyme split | 35S::5′UTR::sfGFPn_Y66-dRibozyme- |
| | | sfGFP linked with GFPuv via | 2nd_sfGFPc-T2A- GFPuv::HSP |
| | | T2A expression | |
| pXYB164 | FIG. 4 | sfGFP linked with GFPuv via | 35S::5′UTR::sfGFP-T2A- GFPuv::HSP |
| | | T2A expression | |
| pXYB084 | FIG. 2 | Plant RNA Vision constructs | 35S:5′UTR::sfGFPn_Y66-P1_loop_01- |
| | | with 123 bp gRNAs together | stem01-gRNA07::HSP - - - |
| | | with RFP input | 35S::5′UTR::gRNA08-stem_02- |
| | | | P1_loop_03-internal guide sequence- |
| | | | Ribozyme-sfGFPn_Y66::HSP - - - |
| | | | 35S::RFP::HSP |
| pXYB085 | FIG. 2 | Plant RNA Vision constructs | 35S::5′UTR::sfGFPn_Y66-P1_loop_01- |
| | | with 164 bp gRNAs together | stem01-gRNA09::HSP -35S::5′ |
| | | with RFP input | UTR:gRNA10-stem_02-P1_loop_03- |
| | | | internal guide sequence- Ribozyme- |
| | | | sfGFPn_Y66::HSP - - - 35S::RFP::HSP |
| pXYB086 | FIG. 2 | Plant RNA Vision constructs | 35S::5′UTR::sfGFPn_Y66- |
| | | with 325 bp gRNAs together | P1_loop_01-stem01-gRNA11::HSP - - - |
| | | with RFP input | 35S::5′UTR::gRNA12-stem_02- |
| | | | P1_loop_03-internal guide sequence- |
| | | | Ribozyme-sfGFPn_Y66::HSP - - - |
| | | | 35S::RFP::HSP |
| pXYB105 | FIG. 7 | GFPuv with 3′ tEvopreq motif | 35S::5′UTR::GFPuv + tEvopreq::HSP |
| | | expression | |
| pXYB106 | FIG. 7 | Ribozyme split GFPuv with | 35S::5′UTR::GFPuvn_Y72-Ribozyme- |
| | | tEvopreq motif expression | 2nd_GFPuvc + tEvopreq::HSP |
| pXYB107 | FIG. 7 | Deactivated ribozyme split | 35S::5′UTR::GFPuvn_Y72-dRibozyme- |
| | | GFPuv tEvopreq motif | 2nd_GFPuvc + tEvopreq::HSP |
| | | expression | |
| pXYB108 | FIG. 7 | GFPuv with 3′ gq2 motif | 35S::5′UTR::GFPuv + gq2::HSP |
| | | expression | |
| pXYB109 | FIG. 7 | Ribozyme split GFPuv with | 35S::5′UTR::GFPuvn_Y72- |
| | | gq2 motif expression | Ribozyme-2nd_GFPuvc + gq2::HSP |
| pXYB110 | FIG. 7 | Deactivated ribozyme split | 35S::5′UTR::GFPuvn_Y72- |
| | | sfGFP gq2 motif expression | dRibozyme-2nd_GFPuvc + gq2::HSP |
| pXYB111 | FIG. 7 | GFPuv with 3′ Triplex motif | 35S::5′UTR::GFPuv + Triplex::HSP |
| | | expression | |
| pXYB112 | FIG. 7 | Ribozyme split GFPuv with | 35S::5′UTR::GFPuvn_Y72-Ribozyme- |
| | | Triplex motif expression | 2nd_GFPuvc + Triplex::HSP |
| pXYB113 | FIG. 7 | Deactivated ribozyme split | 35S::5′UTR::GFPuvn_Y72-dRibozyme- |
| | | GFPuv with Triplex motif | 2nd_GFPuvc + Triplex::HSP |
| | | expression | |
| pXYB176 | FIG. 7 | Ribozyme with IGS2 split | 35S::5′UTR::GFPuvn_Y72-Ribozyme- |
| | | GFPuv | 2nd_GFPuvc + Triplex::HSP |
| | | expression | |
| pXYB177 | FIG. 7 | Deactivated ribozyme with | 35S::5′UTR::GFPuvn_Y72-dRibozyme- |
| | | IGS2 split GFPuv expression | 2nd_GFPuvc + Triplex::HSP |

### Tobacco Leaf Infiltration

The Agrobacterium tumefaciens strain GV3101 harboring the plasmid of interest was infiltrated into the leaves of five- to six-week-old N. benthamiana plants using a 1 mL syringe without a needle as described previously (Li, 2011; Yuan et al., 2022). Briefly, cultures containing the vector of interest were grown in lysogeny broth (LB) medium for 36-48 h and resuspended in agroinfiltration buffer (10 mM MES, PH 5.6, 10 mM MgCl2 and 200 μM acetosyringone) to an optical density of 0.5 at 600 nm (OD600). Bacterial suspensions were incubated for 2-4 h at room temperature and then mixed for co-expression experiments. Agroinfiltrations were carried out through the abaxial surface of the three youngest fully expanded leaves of each plant with a 1-mL needle-free syringe. At least two independent experiments were conducted for each treatment. Each treatment includes six replicates.

Stable Transformation in Arabidopsis thaliana

The Agrobacterium strain ‘GV3101’ was used for the transformation of A. thaliana wild type ‘Col-0’ via the floral dip method with modification as described previously (Yuan et al., 2021). T1 seeds co-transformed with two constructs were selected on K1 medium (1 L; 2.154 g MS, 100 mg myo-inositol, 0.5 g MES, 10 g sucrose, 8 g agar if plates; based on the recommendation of ABRC (Arabidopsis Biological Resource Center) (https://abrc.osu.edu/seed-handling) for Arabidopsis seed germination) with 50 mg/L Kanamycin and 25 mg/L Hygromycin. Seeds were put under dark at 4° C. for 2 days and then germinated for 6-8 days under 100-150 μmol m-2 s-1 fluorescent warm white light, 12 h light/12 h dark, 20° C. and 70% humidity. Then, seedlings were collected for GFP visualization.

### Fluorescence Measurements

The fluorescence of sfGFP and RFP was visualized and imaged using a Zeiss LSM 710 confocal microscope and images were analyzed with the images were analyzed with Fiji software (Schindelin et al., 2012). GFPuv signal was visualized using LIGHTFE UV302D (365 nm) (Yuan et al., 2021).

### Statistical Analysis

The sample average and standard deviation (SD) were calculated from three replicates of each treatment. Significance in all experiments was assessed using a T-Test with an alpha of 0.05. In all cases, resulting P-values of <0.05 for fluorescence intensity of experimental treatment compared to negative control were considered significantly different.

### Western Blot

Leaf discs from infiltrated tobacco leaves were harvested and ground into a fine powder in liquid nitrogen. Total proteins were then extracted by adding 2× Laemmli sample buffer (BioRad) and boiling for 10 min. Cell debris was removed by centrifugation at 16 000 g for 10 min at 4° C. The supernatant was resolved on a 4%-20% SDS-PAGE gel (BioRad) and transferred to polyvinylidene fluoride membranes (Millipore). Anti-GFP polyclonal antibodies (Invitrogen) were used as primary antibodies at a 1:2000 (v/v) dilution. Immunoblots were detected using peroxidase-conjugated anti-rabbit IgG (Sigma-Aldrich) at a 1:7500 (v/v) dilution and ECL substrate (Thermo Scientific) with the BioRad ChemiDoc gel documentation system.

SEQUENCES
SEQ ID NO: 1
sfGFPn Y66
Engineered/Artificial
Nucleic Acid
ATGGTGTTCAATGCTTTTCCCGTTATCCGGATCACATGAAACGGCATGAC
TTTTTCAAGAGTGCCATGCCCGAAGGTTATGTACAGGAACGCACTATATC
TTTCAAAGATGACGGGACCTACAAGACGCGTGCTGAAGTCAAGTTTGAAG
GTGATACCCTTGTTAATCGTATCGAGTTAAAGGGTATTGATTTTAAAGAA
GATGGAAACATTCTTGGACACAAACTCGAGTACAACTTTAACTCACACAA
TGTATACATCACGGCAGACAAACAAAAGAATGGAATCAAAGCTAACTTCA
AAATTCGCCACAACGTTGAAGATGGTTCCGTTCAACTAGCAGACCATTAT
CAACAAAATACTCCAATTGGCGATGGCCCTGTCCTTTTACCAGACAACCA
TTACCTGTCGACACAATCTGTCCTTTCGAAAGATCCCAACGAAAAGCGTG
ACCACATGGTCCTTCTTGAGTTTGTAACTGCTGCTGGGATTACACATGGC
ATGGATGAGCTCTACAAATAA
SEQ ID NO: 2
sfGFPn
Engineered/Artificial
Nucleic Acid
ATGAGCAAAGGAGAAGAACTTTTCACTGGAGTTGTCCCAATTCTTGTTGA
ATTAGATGGTGATGTTAATGGGCACAAATTTTCTGTCCGTGGAGAGGGTG
AAGGTGATGCTACAAACGGAAAACTCACCCTTAAATTTATTTGCACTACT
GGAAAACTACCTGTTCCGTGGCCAACACTTGTCACTACTCTGACCT
SEQ ID NO: 3
eYGFPuv
Engineered/Artificial
Nucleic Acid
ATGACAACCTTCAAAATCGAGTCCCGGATCCACGGCAACCTCAACGGGGA
GAAGTTCGAGTTGGTTGGAGGTGGAGTAGGTGAGGAGGGTCGCCTCGAGA
TTGAGATGAAGACTAAAGATAAACCACTGGCATTCTCTCCCTTCCTGCTG
ACCACTTGCATGGGTTACGGGTTCTACCACTTCGCCAGCTTCCCAAAGGG
GATTAAGAACATCTATCTTCATGCTGCAACGAACGGAGGTTACACCAACA
CCAGGAAGGAGATCTATGAAGACGGCGGCATCTTGGAGGTCAACTTCCGT
TACACTTACGAGTTCAACAAGATCATCGGTGACGTCGAGTGCATTGGACA
TGGATTCCCAAGTCAGAGTCCGATCTTCAAGGACACGATCGTGAAGAGTT
GTCCCACGGTGGACCTGATGTTGCCAATGTCCGGGAACATCATCGCCAGC
TCCTACGCTTACGCCTTCCAACTGAAGGACGGCTCTTTCTACACGGCAGA
AGTCAAGAACAACATAGACTTCAAGAATCCAATCCACGAGTCCTTCTCGA
AGTCAGGGCCCATGTTCACCCACAGACGTGTCGAGGAGACTCTCACCAAG
GAGAACCTTGCCATAGTGGAGTACCAGCAGGTTTTCAACAGCGCCCCAAG
AGACATGTAG
SEQ ID NO: 4
Red Fluorescent Protein
Engineered/Artificial
Nucleic Acid
ATGGCGAGTAGCGAAGACGTTATCAAAGAGTTCATGCGTTTCAAAGTTCG
TATGGAAGGTTCCGTTAACGGTCACGAGTTCGAAATCGAAGGTGAAGGTG
AAGGTCGTCCGTACGAAGGTACCCAGACCGCTAAACTGAAAGTTACCAAA
GGTGGTCCGCTGCCGTTCGCTTGGGACATCCTGTCCCCGCAGTTCCAGTA
CGGTTCCAAAGCTTACGTTAAACACCCGGCTGACATCCCGGACTACCTGA
AACTGTCCTTCCCGGAAGGTTTCAAATGGGAACGTGTTATGAACTTCGAA
GACGGTGGTGTTGTTACCGTTACCCAGGACTCCTCCCTGCAAGACGGTGA
GTTCATCTACAAAGTTAAACTGCGTGGTACCAACTTCCCGTCCGACGGTC
CGGTTATGCAGAAAAAAACCATGGGTTGGGAAGCTTCCACCGAACGTATG
TACCCGGAAGACGGTGCTCTGAAAGGTGAAATCAAAATGCGTCTGAAACT
GAAAGACGGTGGTCACTACGACGCTGAAGTTAAAACCACCTACATGGCTA
AAAAACCGGTTCAGCTGCCGGGTGCTTACAAAACCGACATCAAACTGGAC
ATCACCTCCCACAACGAAGACTACACCATCGTTGAACAGTACGAACGTGC
TGAAGGTCGTCACTCCACCGGTGCTTAA
SEQ ID NO: 5:
Cauliflower mosaic virus 35S Promoter Region
Caulimovirus tessellobrassicae
Nucleic Acid
TGAGACTTTTCAACAAAGGGTAATATCCGGAAACCTCCTCGGATTCCATT
GCCCAGCTATCTGTCACTTTATTGTGAAGATAGTGGAAAAGGAAGGTGGC
TCCTACAAATGCCATCATTGCGATAAAGGAAAGGCCATCGTTGAAGATGC
CTCTGCCGACAGTGGTCCCAAAGATGGACCCCCACCCACGAGGAGCATCG
TGGAAAAAGAAGACGTTCCAACCACGTCTTCAAAGCAAGTGGATTGATGT
GATATCTCCACTGACGTAAGGGATGACGCACAATCCCACTATCCTTCGCA
AGACCCTTCCTCTATATAAGGAAGTTCATTTCATTTGGAGAGAACA
Seq ID NO: 6
Ribozyme
Tetrahymena thermophila
Nucleic Acid
AAAAGTTATCAGGCATGCACCTGGTAGCTAGTCTTTAAACCAATAGATTG
CATCGGTTTAAAAGGCAAGACCGTCAAATTGCGGGAAAGGGGTCAACAGC
CGTTCAGTACCAAGTCTCAGGGGAAACTTTGAGATGGCCTTGCAAAGGGT
ATGGTAATAAGCTGACGGACATGGTCCTAACCACGCAGCCAAGTCCTAAG
TCAACAGATCTTCTGTTGATATGGATGCAGTTCACAGACTAAATGTCGGT
CGGGGAAGATGTATTCTTCTCATAAGATATAGTCGGACCTCTCCTTAATG
GGAGCTAGCGGATGAAGTGATGCAACACTGGAGCCGCTGGGAACTAATTT
GTATGCGAAAGTATATTGATTAGTTTTGGAGTACTCG
Seq ID NO: 7
dRybozyme
engineered/Artificial
Nucleic Acid
AAAAGTTATCAGGCATGCACCTGGTAGCTAGTCTTTAAACCAATAGATTG
CATCGGTTTAAAAGGCAAGACCGTCAAATTGCGGGAAAGGGGTCAACAGC
CGTTCAGTACCAAGTCTCAGGGGAAACTTTGAGATGGCCTTGCAAAGGGT
ATGGTAATAAGCTGACGGACATGGTCCTAACCACGCAGCCAAGTCCTAAG
TCAACAGATCTTCTGTTGATATGGATGCAGTTCACAAACTAAATGTCGGT
CGGGGAAGATGTATTCTTCTCATAAGATATAGTCGGACCTCTCCTTAATG
GGAGCTAGCGGATGAAGTGATGCAACACTGGAGCCGCTGGGAACTAATTT
GTATGCGAAAGTATATTGATTAGTTTTGGAGTACTCG
SEQ ID NO: 8
P2A Self Cleaving Peptide
Teschovirus asilesi
Nucleic Acid
GCTACTAACTTCAGCCTGCTGAAGCAGGCTGGAGACGTGGAGGAGAACCC
TGGACCT
SEQ ID NO: 9
Hairpin 14
Engineered/Artificial
Nucleic Acid
ACGTCGACTCTCGAGTGAGATTGTTGACGGTACCGTATTTT
SEQ ID NO: 10
P1 Loop
Engineered/Artificial
Nucleic Acid
AAATAGCAATATTTACCTTT
SEQ ID NO: 11
P2 Loop_02 Fragment
Engineered/Artificial
Nucleic Acid
TATTTACCTTT
SEQ ID NO: 12
PLT1 gene (AT3G20840)
Arabidopsis thaliana
Nucleic Acid
GAGCTTGGTATATTGATCATACCAAAGTGGTAGTGATTTATTGATTAACC
CAAACACAAAATAAACAGATTTGACTCAAAAAGAAGAAAATGAATTCTAA
CAACTGGCTTGGCTTTCCTCTTTCACCGAACAACTCTTCTTTGCCTCCTC
ATGAATACAACCTTGGCTTGGTCAGCGACCATATGGACAACCCTTTTCAA
ACACAAGAGTGGAATATGATCAATCCACACGGTGGAGGAGGAGATGAAGG
AGGAGAGGTTCCAAAAGTGGCCGATTTTCTCGGTGTGAGCAAACCGGACG
AAAACCAATCCAACCACCTAGTAGCTTACAACGACTCAGACTACTACTTC
CATACCAATAGCTTGATGCCTAGCGTCCAATCAAACGATGTCGTTGTAGC
AGCTTGTGACTCCAATACTCCTAACAACAGTAGCTATCATGAGCTTCAAG
AGAGTGCTCACAATCTACAGTCACTTACTTTGTCCATGGGGACCACCGCT
GGTAATAATGTTGTAGACAAAGCTTCACCATCCGAGACCACCGGGGATAA
CGCTAGCGGTGGAGCACTAGCCGTTGTTGAGACGGCCACGCCAAGACGTG
CATTGGACACTTTCGGACAACGAACCTCGATCTATCGTGGTGTCACAAGA
CATCGATGGACTGGTCGATATGAGGCTCATCTATGGGATAATAGTTGTAG
AAGGGAAGGCCAGTCTAGGAAAGGAAGACAAGTTTACTTGGGTGGATATG
ACAAAGAAGATAAAGCAGCAAGATCATATGATCTAGCTGCACTTAAGTAC
TGGGGTCCTTCAACTACTACTAATTTCCCCATTACAAACTACGAGAAAGA
AGTAGAGGAAATGAAGCACATGACGAGACAAGAGTTCGTGGCTGCCATTA
GAAGGAAAAGTAGTGGATTTTCGAGAGGCGCTTCGATGTATCGAGGAGTT
ACAAGGCATCACCAACATGGAAGATGGCAAGCAAGGATCGGCCGAGTCGC
CGGAAACAAAGACCTCTACTTGGGAACTTTTAGCACTGAGGAAGAAGCAG
CAGAAGCTTACGATATAGCTGCAATAAAGTTTAGAGGACTTAATGCAGTG
ACCAACTTCGAGATCAACCGGTACGACGTGAAAGCCATTCTAGAGAGTAG
CACTCTTCCCATCGGAGGAGGCGCAGCTAAACGGCTCAAAGAAGCTCAAG
CTCTTGAGTCTTCAAGGAAACGCGAGGCGGAGATGATAGCCCTTGGTTCA
AGTTTCCAGTACGGTGGTGGCTCGAGCACAGGCTCTGGCTCCACCTCATC
AAGACTTCAGCTTCAACCTTACCCTCTAAGCATTCAACAACCATTAGAGC
CTTTTCTATCTCTTCAGAACAATGACATCTCTCATTACAACAACAACAAT
GCTCACGATTCCTCCTCTTTTAATCACCATAGCTATATCCAGACACAACT
TCATCTCCACCAACAGACCAACAATTACTTGCAGCAACAGTCGAGCCAGA
ACTCTCAGCAGCTCTACAATGCGTATCTTCATAGCAATCCGGCTCTGCTT
CATGGACTTGTCTCTACCTCTATCGTTGACAACAATAATAACAATGGAGG
CTCTAGTGGGAGCTACAACACTGCAGCATTTCTTGGGAACCACGGTATTG
GTATTGGGTCCAGCTCGACTGTTGGATCGACCGAGGAGTTTCCAACCGTT
AAAACAGATTACGATATGCCTTCCAGTGATGGAACCGGAGGGTATAGTGG
TTGGACCAGTGAGTCTGTTCAGGGGTCAAACCCTGGTGGTGTTTTCACTA
TGTGGAATGAGTAAACAAGGATCTCTTTCTTGCGGCACAAGGAATGGGTC
TGAATAGTAACTATTTTAATCTCTACTCTCAATTTTTATTTCTTTCTTGT
CTTGGAAGTAATAGGATCAATTTAGAGGGGGCCTTTTGGTAGAAGAATTT
AGGTGACGCAAGGAGTTCTTTCTATATTTACGGCGAAGGTATTCTCAGAC
TGGTTAAATGGTTCTTGAGGGTTAGGGTTTTATGGGTACTCAATTAAGTT
TGTGTACCCAAATTTTGTTTACAGCTAACCCTTTTGACTAATTTCCAAAT
TTGTAATTCGTTTATAAAGATGTTTTCTCTAGCTCTT
SEQ ID NO: 13
tobacco rattle virus Cap protein
Tobravirus tabaci
Nucleic Acid
ATGGGAGATATGTACGATGAATCATTTGACAAGTCGGGCGGTCCTGCTGA
CTTGATGGACGATTCTTGGGTGGAATCAGTTTCGTGGAAAGATCTGTTGA
AGAAGTTACACAGCATAAAATTTGCACTACAGTCTGGTAGAGATGAGATC
ACTGGGTTACTAGCGGCACTGAATAGACAGTGTCCTTATTCACCATATGA
GCAGTTTCCAGATAAGAAGGTGTATTTCCTTTTAGACTCACGGGCTAACA
GTGCTCTTGGTGTGATTCAGAACGCTTCAGCGTTCAAGAGACGAGCTGAT
GAGAAGAATGCAGTGGCGGGTGTTACAAATATTCCTGCGAATCCAAACAC
AACGGTTACGACGAACCAAGGGAGTACTACTACTACCAAGGCGAACACTG
GCTCGACTTTGGAAGAAGACTTGTACACTTATTACAAATTCGATGATGCC
TCTACAGCTTTCCACAAATCTCTAACTTCGTTAGAGAACATGGAGTTGAA
GAGTTATTACCGAAGGAACTTTGAGAAAGTATTCGGGATTAAGTTTGGTG
GAGCAGCTGCTAGTTCATCTGCACCGCCTCCAGCGAGTGGAGGTCCGATA
CGTCCTAATCCC
SEQ ID NO: 14
Anti-CRISPR AcrIIA5
Engineered/Artificial
Nucleic Acid
ATGGCCTATGGCAAGTCTCGTTATAATTCCTATAGGAAACGTAGCTTCAA
CCGATCTAACAAGCAACGTAGAGAATACGCACAGGAGATGGATCGACTTG
AGAAAGCATTCGAAAACCTGGACGGGTGGTACCTTTCCTCAATGAAGGAT
AGCGCCTACAAGGATTTTGGTAAGTACGAAATCCGATTGTCTAACCACTC
AGCCGACAATAAATATCATGATCTCGAAAATGGGCGATTGATCGTGAACA
TCAAGGCTTCAAAACTCAATTTTGTCGATATTATCGAAAATAAACTTGAT
AAGATCATCGAGAAGATCGACAAGCTCGACCTGGATAAGTATAGATTTAT
TAACGCAACCAATCTCGAACATGACATTAAGTGCTACTACAAAGGCTTCA
AGACGAAAAAAGAGGTGATT
SEQ ID NO: 15
gRNA_01
Engineered/Artificial
Nucleic Acid
GGAGGAGTCCTGGGTAACGGTAACAACACCACCGTCTTCGA
SEQ ID NO: 16
gRNA_02
Engineered/Artificial
Nucleic Acid
TGGTACCACGCAGTTTAACTTTGTAGATGAACTCACCGTCT
SEQ ID NO: 17
gRNA_03
Engineered/Artificial
Nucleic Acid
GGAGGAGTCCTGGGTAACGGTAACAACACCACCGTCTTCGAAGTTCATAA
CACGTTCCCATTTGAAACCTTCCGGGAAGGAC
SEQ ID NO: 18
gRNA_04
Engineered/Artificial
Nucleic Acid
CATGGTTTTTTTCTGCATAACCGGACCGTCGGACGGGAAGTTGGTACCAC
GCAGTTTAACTTTGTAGATGAACTCACCGTCT
SEQ ID NO: 19
gRNA_05
Engineered/Artificial
Nucleic Acid
CCGGTTTGCTCACACCGAGAAAATCGGCCACTTTTGGAACCTCTCCTCCT
TCATCTCCTCCTCCACCGTGTGGATTGATCAT
SEQ ID NO: 20
gRNA_06
Engineered/Artificial
Nucleic Acid
TTGATTGGACGCTAGGCATCAAGCTATTGGTATGGAAGTAGTAGTCTGAG
TCGTTGTAAGCTACTAGGTGGTTGGATTGGTT
SEQ ID NO: 21
gRNA_07
Engineered/Artificial
Nucleic Acid
GGAGGAGTCCTGGGTAACGGTAACAACACCACCGTCTTCGAAGTTCATAA
CACGTTCCCATTTGAAACCTTCCGGGAAGGACAGTTTCAGGTAGTCCGGG
ATGTCAGCCGGGTGTTTAACGTA
SEQ ID NO: 22
gRNA_08
Engineered/Artificial
Nucleic Acid
CCGTCTTCCGGGTACATACGTTCGGTGGAAGCTTCCCAACCCATGGTTTT
TTTCTGCATAACCGGACCGTCGGACGGGAAGTTGGTACCACGCAGTTTAA
CTTTGTAGATGAACTCACCGTCT
SEQ ID NO: 23
gRNA_09
Engineered/Artificial
Nucleic Acid
GGAGGAGTCCTGGGTAACGGTAACAACACCACCGTCTTCGAAGTTCATAA
CACGTTCCCATTTGAAACCTTCCGGGAAGGACAGTTTCAGGTAGTCCGGG
ATGTCAGCCGGGTGTTTAACGTAAGCTTTGGAACCGTACTGGAACTGCGG
GGACAGGATGTCCC
SEQ ID NO: 24
gRNA_10
Engineered/Artificial
Nucleic Acid
CTTTCAGTTTCAGACGCATTTTGATTTCACCTTTCAGAGCACCGTCTTCC
GGGTACATACGTTCGGTGGAAGCTTCCCAACCCATGGTTTTTTTCTGCAT
AACCGGACCGTCGGACGGGAAGTTGGTACCACGCAGTTTAACTTTGTAGA
TGAACTCACCGTCT
SEQ ID NO: 25
gRNA_11
Engineered/Artificial
Nucleic Acid
GGAGGAGTCCTGGGTAACGGTAACAACACCACCGTCTTCGAAGTTCATAA
CACGTTCCCATTTGAAACCTTCCGGGAAGGACAGTTTCAGGTAGTCCGGG
ATGTCAGCCGGGTGTTTAACGTAAGCTTTGGAACCGTACTGGAACTGCGG
GGACAGGATGTCCCAAGCGAACGGCAGCGGACCACCTTTGGTAACTTTCA
GTTTAGCGGTCTGGGTACCTTCGTACGGACGACCTTCACCTTCACCTTC
SEQ ID NO: 26
Heat Shock Protein terminator
Arabidopsis thaliana
Nucleic acid
ATATGAAGATGAAGATGAAATATTTGGTGTGTCAAATAAAAAGCTTGTGT
GCTTAAGTTTGTGTTTTTTTCTTGGCTTGTTGTGTTATGAATTTGTGGCT
TTTTCTAATATTAAATGAATGTAAGATCTCATTATAATGAATAAACAAAT
GTTTCTATAATCCATTGTGAATGTTTTGTTGGATCTCTTCTGCAGCATAT
AACTACTGTATGTGCTATGGTATGGACTATGGAATATGATTAAAGATAAG
ATGGGCTCATAGAGTAAAACGAGGCGAGGGACCTATAAACCTCCCTTCAT
CATGCTATTTCATGATCTATTTTATAAAATAAAGATGTAGAAAAAAGTAA
GCGTAATAACCGCAAAACAAATGATTTAAAACATGGCACATAATGAGGAG
ATTAAGTTCGGTTTACGTTTATTTTAGTACTAATTGTAACGTGAGACTAC
GTATCGGGAATCGCCTAATTAAAGCATTAATGCGAACCTGATTAGATTCA
CCGACCCTCCTATCGTGTCGACCTTTCTGTTTCTTAGAATTTTTTGGTAG
TCTATGTACTAATAATGTCAGCTTCGTATTTATTTCATAAGCAATTTGCA
TTTGCAATTTGTTTTTTACTTTTATTTTTATTGTATTGTGGAATGTGGAC
TCGTACCAACATGAAGTTATATACCACCAAAAAAATTACAGTTAGTCAAA
AGATTCACGAGTGAGAGCTACTTATGATTGTCTTTTACGTATATGTCTAA
TTGTCTATTTGCTCAATAATCTTTGTACTTTCTTTTGTCGTTGATAAAAT
CACAAAGTTCCAAAAGTAATCGAATGATTTGCTTTTAAGAAAAGAAGAGC
TCAATAATTCAACATATATCTGTACACA
SEQ ID NO: 27
5′ Untranslated Region cold regulated 47 gene
Arabidopsis thaliana
Nucleic acid
CAAACATTACTCAT
TCACAAAACCATCTTAAAGCAACTACACAAGTCTTGAAATTTTCTCATAT
TTTCTATTTACTATATAAACTTTTAATCAAATCAAGATTAAAG
SEQ ID NO: 28
P2A self-cleaving peptide
Teschovirus asilesi
Amino acid
ATNFSLLKQAGDVEENPGP
SEQ ID NO: 29
E2A self-cleaving peptide
Aphthovirus burrowsi
Amino acid
QCTNYALLKLAGDVESNPGP
SEQ ID NO: 30
F2A self-cleaving peptide
Aphthovirus vesiculae
Amino acid
VKQTLNFDLLKLAGDVESNPGP
SEQ ID NO: 31
T2A self-cleaving peptide
Alphapermutotetravirus thoseae
Amino acid
EGRGSLLTCGDVEENPGP

### REFERENCES

Alamos, S., et al. (2021) Quantitative imaging of RNA polymerase II activity in plants reveals the single-cell basis of tissue-wide transcriptional dynamics. Nat. Plants, 7, 1037-1049.
Bai, J. Y., et al. (2020) A protein-independent fluorescent RNA aptamer reporter system for plant genetic engineering. Nat. Commun. 11, 3847.
Bleckmann, A. and Dresselhaus, T. (2016) Fluorescent whole-mount RNA in situ hybridization (F-WISH) in plant germ cells and the fertilized ovule. Methods, 98, 66-73.
Brophy, J. A. N., et al . . . (2022) Synthetic genetic circuits as a means of reprogramming plant roots. Science, 377, 747-751.
Carrier, T. A. and Keasling, J. D. (1999) Library of synthetic 50 secondary structures to manipulate mRNA stability in Escherichia coli. Biotechnol. Prog. 15, 58-64.
Cermak, T., et al. (2017) A multipurpose toolkit to enable advanced genome engineering in plants. Plant Cell, 29, 1196-1217.
Gambill, L., et al. (2023) A split ribozyme that links detection of a native RNA to orthogonal protein outputs. Nat. Commun. 14, 543.
Großkinsky, D. K., et al . . . (2015) Plant phenomics and the need for physiological phenotyping across scales to narrow the genotype-to-phenotype knowledge gap. J. Exp. Bot. 66, 5429-5440.
Hasegawa, S., Choi, J. W. and Rao, J. (2004) Single-cell detection of transsplicing ribozyme in vivo activity. J. Am. Chem. Soc. 126, 7158-7159.
Hasegawa, S., Gowrishankar, G. and Rao, J. (2006) Detection of mRNA in mammalian cells with a split ribozyme reporter. Chembiochem, 7, 925-928.
Islam, M. T., et al. (2024) Advances in the application of single-cell transcriptomics in plant systems and synthetic biology. Biodes. Res. 6, 29.
K€ohler, U., et al . . . (1999) Trans-splicing ribozymes for targeted gene delivery. J. Mol. Biol. 285, 1935-1950.
Li, λ. (2011) Infiltration of Nicotiana benthamiana protocol for transient expression via Agrobacterium. Bio-protocol, 1(14), e95.
Liang, J. C., Bloom, R. J. and Smolke, C. D. (2011) Engineering biological systems with synthetic RNA molecules. Mol. Cell, 43, 915-926.
Liu, D., et al. (2019) CRISPR/Cas9-mediated targeted mutagenesis for functional genomics research of crassulacean acid metabolism plants. J. Exp. Bot. 70, 6621-6629.
Liu, Y., et al . . . (2020) Functional analysis of the teosinte branched 1 gene in the tetraploid switchgrass (Panicum virgatum L.) by CRISPR/Cas9-directed mutagenesis. Front. Plant Sci. 11, 572193.
Liu, Y., et al. (2022) Biological and molecular components for genetically engineering biosensors in plants. Biodes. Res. 2022, 9863496.
Liu, D., et al. (2024) Heritable gene editing in tomato through viral delivery of isopentenyl transferase and single-guide RNAs to latent axillary meristematic cells. Proc. Natl. Acad. Sci. USA, 121, e2406486121.
Martin, L., et al. (2013) Catalyzing plant science research with RNA-seq. Front. Plant Sci. 4, 66.
Nawy, T., et al. (2005) Transcriptional profile of the Arabidopsis root quiescent center. Plant Cell, 17, 1908-1925.
Rogers, C. S., et al. (2002) Functional repair of a mutant chloride channel using a trans-splicing ribozyme. J. Clin. Invest. 110, 1783-1789.
Ryu, K. H., et al. (2019) Single-cell RNA sequencing resolves molecular relationships among individual plant cells. Plant Physiol. 179, 1444-1456.
Schindelin, J., et al. (2012) Fiji: an open-source platform for biological image analysis. Nat. Methods, 9, 676-682.
Selinidis, M. A., et al. (2024) Ribozymemediated gene-fragment complementation for nondestructive reporting of dna transfer within soil. ACS Synth. Biol. 13, 3539-3547.
Yang, X., et al. (2020) Plant biosystems design research roadmap 1.0. Biodes. Res. 2020, 8051764.
Yuan, G. L., et al. (2021) Expanding the application of a UV-visible reporter for transient gene expression and stable transformation in plants. Hortic. Res. 8, 234.
Yuan, G. L., et al. (2022) An intein-mediated split-nCas9 system for base editing in plants. ACS Synth. Biol. 11, 2513-2517.
Yuan, G., et al. (2023) Split selectable marker systems utilizing inteins facilitate gene stacking in plants. Commun. Biol. 6, 567.
Zhao, F., et al. (2021) An optimized protocol for stepwise optimization of real-time RTPCR analysis. Hortic. Res. 8, 179.

1. A split ribozyme biosensor system, comprising:
a first expression cassette comprising a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and
a second expression cassette comprising a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein;
wherein the first and second fragments of the ribozyme together provide a full-length sequence of the ribozyme,
wherein the first and second fragments of the protein together provide a full-length sequence of the protein; and
wherein upon transcription in a plant and when a target RNA is present in the plant, the first guide RNA sequence and the second guide RNA sequence bind to the target RNA, and bring the two ribozyme fragments together so that the ribozyme removes itself from flanking sequences to allow formation of an mRNA encoding the full length sequence of the first protein.

2. The biosensor system of claim 1, wherein the first protein is a reporter protein.

3. The biosensor system of claim 1, wherein the first protein is transiently expressed.

4. The biosensor system of claim 1, wherein the first protein is expressed after the two guide RNAs binding to the target RNA.

5. The biosensor system of claim 1, wherein the ribozyme comprises an internal guide sequence (IGS) which begins with a guanine and the remainder of the IGS is the reverse complement to the first five base pairs of a P1 helix.

6. The biosensor system of claim 5, wherein:
the first fragment of the ribozyme is inserted immediately downstream of an uracil positioned within the nucleotide sequence encoding the first protein such that insertion of the ribozyme creates a fragmentation in the nucleotide sequence encoding the first protein, and
wherein each fragment of the nucleotide sequence does not encode a functional protein.

7. The biosensor system of claim 1, wherein the first polynucleotide sequence further comprises a promoter sequence and a 5′ untranslated sequence that initiates gene expression in the plant and is operably linked to the 5′ of the nucleotide sequence encoding the first fragment of the first protein.

8. The biosensor system of claim 1, wherein the first and second polynucleotide sequences are either:
contiguous and under control of a same promoter; or
under the control of separate promoter sequences and 5′ untranslated sequences.

9. (canceled)

10. The biosensor system of claim 1, wherein both the first and second polynucleotide sequences each comprise a terminator sequence that signals the RNA polymerase to stop transcription in the plant.

11. The biosensor system of claim 1, wherein both the first guide RNA sequence and the second guide RNA sequence range from about 40 to 325 nucleotides in length.

12. The biosensor system of claim 11,
wherein the first RNA guide sequence and/or the second RNA guide sequence;
each range from about 60 to 170 nucleotides in length; or
each range from about 80 to 125 nucleotides in length; or
are each:
about 41 nucleotides in length;
about 82 nucleotides in length;
about 123 nucleotides in length;
about 164 nucleotides in length; or
about 325 nucleotides in length;
and
wherein the first RNA guide sequence and the second RNA guide sequence are either the same length or different lengths.

13.-14. (canceled)

15. The biosensor system of claim 1, wherein the second polynucleotide further comprises a nucleotide sequence encoding a self-cleaving peptide linked to a second protein, wherein upon translation, the first protein and the second protein are separated.

16. The biosensor system of claim 15, wherein the second protein is a reporter protein, and the first and second reporter proteins are different reporter proteins.

17. (canceled)

18. The biosensor system of claim 15, wherein the self-cleaving peptide is P2A, E2A, F2A, or T2A.

19. The biosensor system of claim 1, further comprising a third polynucleotide sequence comprising a promoter sequence, a 5′ untranslated sequence, a nucleotide sequence encoding a third protein, and a terminator sequence.

20. The biosensor system of claim 19, wherein the third protein is a reporter protein.

21. The biosensor system of claim 2, wherein the reporter protein is a fluorescent protein reporter or bioluminescence reporter protein (luciferase).

22. A method for examining in vivo RNA expression in plants, plant cells, or plant tissues, the method comprising:
introducing the biosensor system of claim 1 into a plant, plant cell, or plant tissue to create a genetically modified plant, plant cell, or plant tissue.

23. The method of claim 22, wherein the first protein of the biosensor system is a reporter protein, the method further comprising detecting the reporter protein as indicative of the presence of the target RNA.

24. The method of claim 22, wherein the second protein of the biosensor system is a reporter protein, the method further comprising detecting the reporter protein as indicative of the presence of the target RNA.

25. The method of claim 23, wherein the detecting the reporter protein occurs through optical imaging or electrochemical imaging.

26. (canceled)

27. The method of claim 22, further comprising subjecting the transgenic plant to experimental conditions before the step of detecting the reporter protein, wherein the experimental conditions comprise abiotic stress, biological stress, improved growth conditions.

28. A genetically modified plant, plant cell, or plant tissue comprising the biosensor system of claim 1.

29. A kit comprising:
a first polynucleotide sequence comprising from 5′ to 3′: a nucleotide sequence encoding a first fragment of a first protein, a nucleotide sequence encoding a first fragment of a ribozyme, and a nucleotide sequence encoding a first guide RNA sequence; and
a second polynucleotide sequence comprising from 5′ to 3′: a nucleotide sequence encoding a second guide RNA sequence, a nucleotide sequence encoding a second fragment of the ribozyme, and a nucleotide sequence encoding a second fragment of the first protein;
wherein the polynucleotide sequences can be delivered into plants or plant cells.

30. The kit of claim 29, further comprising a third nucleotide sequence encoding a second protein.

31. (canceled)
